(19) United States
(12) Patent Application Publication
(10) Pub. No.: US 2025/0316387 A1
(43) Pub. Date: Oct. 9, 2025

(54) MULTI-MODAL MACHINE LEARNING APPROACHES FOR PREDICTING CANCER TYPE AND GLEASON GRADE LEVERAGING PUBLIC TCGA DATA

(71) Applicant: Roche Molecular Systems, Inc., Pleasanton, CA (US)

(72) Inventors: Mohammad Ashtari, Ottawa (CA); Ofir Etz Hadar, Holon (IL); Jacob Gildenblat, Holon (IL); Michael King, Munich (DE); Eldad Klaiman, Starnberg (DE); Antoaneta Petkova Vladimirova, Mountain View, CA (US); Jakub Witkowski, Zielonka (PL); Christian Wohlfart, Penzberg (DE)

(73) Assignee: Roche Molecular Systems, Inc., Pleasanton, CA (US)

(21) Appl. No.: 19/200,021

(22) Filed: May 6, 2025

Related U.S. Application Data
(63) Continuation of application No. PCT/EP2023/081973, filed on Nov. 15, 2023.
(60) Provisional application No. 63/383,951, filed on Nov. 16, 2022.

(51) Int. Cl. G16H 50/20; G16H 30/40

(52) U.S. Cl. CPC G16H 50/20; G16H 30/40

(57) ABSTRACT

The invention relates to a method of diagnosing or determining the prognosis of cancer in a patient, the method comprising processing, using RNA-sequencing and a first machine learning model, the genomic data of the patient to determine at least one of a first cancer type or degree of cancer and processing, using histopathology and a second machine learning model, the biopsy image data of the patient to determine at least one of a second cancer type or degree of cancer and comparing the determined first type or degree of cancer with the determined second type or degree of cancer and correlating the two.

### CROSS-REFERENCE TO RELATED APPLICATIONS

This application is a continuation of International Application No. PCT/EP2023/081973, filed Nov. 15, 2023, which claims the benefit of U.S. Provisional Application No. 63/383,951, filed Nov. 16, 2022, the disclosures of which are hereby incorporated by reference in their entirety

### FIELD OF THE INVENTION

Early and accurate diagnosis of most diseases is vital for optimising treatment regimes, lowering healthcare costs and ultimately for improving health outcomes. This is particularly the case for serious and life-threatening diseases such as cancer, for which the treatment itself can be severely debilitating. The earlier and more accurately a cancer diagnosis can be made, the less likely it is that the cancer has metastasized and the less severe the treatment needs to be. Current techniques for diagnosing cancers may involve imaging such as mammography or diagnostic tests to determine the presence of biomarkers in the blood or in tissue samples, such as the prostate specific antigen test.

Despite the progress made with such diagnostic methods, there is still the danger of false positive or false negative results leading either to the administration of treatments which are unnecessary or to the failure to identify the cancer until medical intervention may not be successful. There is thus the need for more accurate, faster and earlier diagnosis of cancers of all types.

To better understand the complex and challenging nature of diseases such as cancer and for improved diagnosis, it may require the combination of multiple data modalities, such as histopathological images and omics data such as RNA-seq. By integrating these heterogeneous but complementary data, a multimodal approach unites both worlds and could achieve better synergistic results compared to using a single modality. The growing availability of large datasets such as The Cancer Genome Atlas (TCGA) with more than 10000 patients makes it possible to combine different modalities to train machine learning algorithms which offers great potential to address challenging cancer related research. In this invention machine learning approaches are used within an open-source framework in order to leverage multimodality (Histopathology Whole Slide Images (WSI) and Genomics/RNA-seq to build predictive AI models such as for diagnosing cancer type and prostate Gleason score, among other diagnoses, and provide a significant quality control step pertaining to such diagnosis utilizing other modalities.

### OBJECT OF THE INVENTION

It is an object of the invention to develop a machine learning model to classify cancers. Another object is to determine which data modalities are best suited to diagnose and/or prognose different cancers. A further object is to develop a machine learning model based on Whole Slide Imaging and Genomics RNA-sequence profiles, for the classification and prediction of cancers. A still further object is to provide an improved diagnostic/prognostic method for cancers, which provides earlier and more accurate diagnosis of cancers and survival rates. Still further objects of the invention are to provide a computer system and/or a computer program for diagnosing or determining the prognosis of cancer in a patient based on a machine learning model.

### SUMMARY OF THE INVENTION

The present invention provides a method of diagnosing or determining the prognosis cancer in a patient, the method comprising:
receiving genomic data of a patient;
receiving biopsy image data of the patient;
processing, using RNA-sequencing and a first machine learning model, the genomic data of the patient to determine at least one of a first cancer type or degree of cancer;
processing, using histopathology and a second machine learning model, the biopsy image data of the patient to determine at least one of a second cancer type or degree of cancer;
comparing the determined first type or degree of cancer with the determined second type or degree of cancer;
in response to determining a level of correlation between the determined first cancer type or degree and the second determined cancer type or degree, generating an output diagnosing or determining the prognosis cancer in the patient as the first cancer type or degree; or
in response to determining that the determined first cancer type or degree and the determined second cancer type or degree do not have the level of correlation, generating an output indicating that the diagnosing or determining the prognosis is undetermined.

The first machine learning model may comprise at least one of a support vector machine (SVM) or gradient boosting decision tree (GBDT). The second machine learning model comprises attention-based multiple instance learning (Attention MIL) or Resnet 18. In some cases a linear SVM model may be combined with a Resnet 18 model by multiplying the probability scores of each single-modality model.

The genomic data may be RNA sequence data. In particular, the genomic data may be RNA sequences derived from protein-encoding genes.

The method can diagnose or determine the prognosis of at least one of cervical squamous cell carcinoma and endocervical adenocarcinoma (CESC), cholangiocarcinoma (CHOL), uterine carcinosarcoma (UCS), or Gleason score.

The method can predict Luad/Lusc overall survival rate.

Determining the level of correlation may comprise determining that the first and second types or degrees of cancer are the same and that an F1 score for the first machine learning model with respect to the first type or degree of cancer exceeds a first predetermined threshold and that an F1 score for the second machine learning model with respect the second type or degree of cancer exceeds a second predetermined threshold. The F1 score may be at least 90%, or at least 93% or at least 95% or at least 98%.

The invention also relates to a non-transitory computer-readable storage medium storing one or more computer programs configured to be executed by one or more processing units at a computer comprising instructions for:
receiving genomic data of a patient;
receiving biopsy image data of the patient;
processing, using RNA-sequencing and a first machine learning model, the genomic data of the patient to determine at least one of a first cancer type or degree of cancer;
processing, using histopathology and a second machine learning model, the biopsy image data of the patient to determine at least one of a second cancer type or degree of cancer;
comparing the determined first type or degree of cancer with the determined second type or degree of cancer; or
   in response to determining a level of correlation between the determined first cancer type or degree and the second determined cancer type or degree, generating an output diagnosing or determining the prognosis of cancer in the patient as the first cancer type or degree; or
   in response to determining that the determined first cancer type or degree and the determined second cancer type or degree do not have the level of correlation, generating an output indicating that the diagnosing or determining the prognosis is undetermined. The invention also provides a computer system for diagnosing or determining the prognosis of cancer in a patient, the computer system comprising one or more processors, memory to store one or more computer programs, the computer programs comprising instructions for
receiving genomic data of a patient;
receiving biopsy image data of the patient;
processing, using RNA-sequencing and a first machine learning model, the genomic data of the patient to determine at least one of a first cancer type or degree of cancer;
processing, using histopathology and a second machine learning model, the biopsy image data of the patient to determine at least one of a second cancer type or degree of cancer;
comparing the determined first type or degree of cancer with the determined second type or degree of cancer;
   in response to determining a level of correlation between the determined first cancer type or degree and the second determined cancer type or degree, generating an output diagnosing or determining the prognosis of cancer in the patient as the first cancer type or degree; or
   in response to determining that the determined first cancer type or degree and the determined second cancer type or degree do not have the level of correlation, generating an output indicating that the diagnosing or determining the prognosis is undetermined.

### BRIEF DESCRIPTION OF THE DRAWINGS

FIG. 1: Auto-sklearn pipeline.

FIG. 2: Confusion matrix for gender prediction (normalised left; normal right).

FIG. 3. SHAP values for gender prediction

FIG. 4: t-SNE visualisation for all cancer types using FPKM data.

FIG. 5: Class distribution of train and test set for each cancer type.

FIG. 6: Normalized confusion matrix for the LDA classification for all 30 cancer types.

FIG. 7: Confusion matrix for the LDA classification for all 30 cancer types.

FIG. 8. SHAP values for cancer type prediction (importance TOP 20 features) and the class wise influence for the genes:—RDH11—retinol dehudrogenase 11; QKI—QKI, KH domain containing RNA binding; C5—complement 5; TMEM241—transmembrane protein 241; NAP1L5—nucleosome assembly protein 1 like 5; SLC12A2—solute carrier family 12 member 2; GNA15—G protein subunit alpha 15; NECTIN1—nectin cell adhesion molecule 1; TMOD2—tropomodulin 2; FAM49A—CYFIP related Rac 1 interactor A; F11R—junctional adhesion molecule A; GATA2—GATA-binding factor 2; TMEM101—transmembrane protein 101; STAMBPL1—STAM binding protein like 1; UQCRHL—cytochrome b-c1complex subunit 6; PIAS1—protein inhibitor of activated STAT 1; ASRGL1—asparaginase and isopartyl peptidase 1; GYPC—glycophorin C; ANXA4—annexin IV; H3F3A—histone H3.3.

FIGS. 9A-9G. Charts of feature impact for each cancer type on the prediction.

FIG. 10: Confusion matrix for gender prediction (normalized left; normal right).

FIG. 11: Confusion matrix for gender prediction (normalized left; normal right)

FIG. 12. SHAP values for primary gleason score prediction (importance TOP 20 features) and the class wise influence for the genes:—FLCN—folliculin; RNF10—ring finger protein 10; ARMCX6—armadillo repeat containing X-linked 6; GLO1—glyoxalase 1; SPRYD7—spry domain containing 7; CD44—CD44 molecule; AFG1L—AFG1 like ATPase; GDPGP1—GDP-D-glucose phosphorylase 1; MAGOHB—mago homolog B; IFT74—intraflagellar transport 74; ATXN10—ataxin 10; TMEM17—transmembrane protein 17; ZNF197—zinc finger protein 197; PIGZ—phosphatidylinositol glycan anchor biosynthesis class Z; CHST12—carbohydrate sulfotransferase 12; AAGAB—alpha and gamma adaptin binding protein; ALG3—alpha-1,3-mannosyltransferase; PTP4A2—protein tyrosine phosphatase 4A2; UQCRHL—cytochrome b-c1complex subunit 6; PIK3R3—phosphoinositide-3-kinase regulatory subunit 3.

FIG. 13. Feature impact for each Gleason pattern on the prediction.

FIG. 14. Visualisation of LUAD and LUSC embeddings

FIG. 15. Visualisation of UAMP embeddings

FIG. 16. Confusion matrix for prediction of cancer types.

FIG. 17 Confusion matrix for cancer type.

FIG. 18. Visualisation of all UAMP embeddings.

FIG. 19. Confusion matrix for Gleason patterns 3/4/5.

FIG. 20. Confusion matrix for Gleason pattern3 vs 4.

### DETAILED DESCRIPTION

It is important for any machine learning model that evaluation metrics are used to determine the effectiveness of the machine learning model. The F1 score is a fundamental metric for evaluating classification models. Using multiple metrics to evaluate the performance of a model is a common practice in machine learning tasks, since the model can give good outcomes on one metric and perform suboptimally in another. Any model therefore needs to find a balance between the various metrics. The present invention relates to a classification model which requires metrics like accuracy, precision, recall, F1 score and area under the ROC curve. A model dealing with cancer diagnostics and prognostics has to deal with true positives, true negatives, false positives when the events are wrongly predicted as positive when in fact they are negative, and false negatives in which an event is wrongly predicted as negative when in fact it is positive.

The accuracy metric calculates the overall prediction correctness by dividing the number of correctly predicted positive and negative events by the total number of events. The precision metric determines the quality of positive predictions by measuring their correctness, and is the number of true positive outcomes divided by the sum of the true positive and false positive predictions. Recall, which is sometimes called sensitivity, measures the model's ability to detect positive events correctly and is the percentage of accurately predicted positive events out of all actual positive events. The F1 score can be described as the harmonic mean of the precision and recall of a classification model. So in this invention, recall is a measure of how many of the cancer slides the model can correctly predict where is the precision value is a measure of how many of the slides where cancer was predicted we're actually correct. The two metrics contribute equally to the score ensuring that the F1 metric correctly indicates the reliability of a model. The F1 score varies between zero and one, with a score of 1 representing a flawless result. The area under the curve is a measure of how the model's predictions are correctly ranked between two categories. In other words is the model able to give a higher value to an example from one category than to an example from another category.

In this invention the machine model analyzes whole slide images. Such digitized images may represent substantial amounts of data. With a supervised learning model, precise regions of the image are annotated with a label (e.g., cancer type or Gleason score) and a model is created that learns to detect these regions. In a weakly supervised learning model a label is assigned to the whole slide image but precise regions are not annotated. This invention can verify whether a model successfully predicts such a label. The whole slide image is divided into small areas and models are used to aggregate all the information to create a slide level prediction. This is then used in a multi modal model in which the imaging model is combined with an RNA model to determine if there can be an improvement in the accuracy of the cancer diagnosis or prognosis.

Matched WSI and RNA-Seq profiles from TCGA, including 11093 samples and 30 cancer types were used to develop a pancancer classification model using both modalities. For prostate Gleason score prediction 401 patients were available. Both datasets were split into a train (70%) and test (30%) components. A late fusion approach was used where the RNA-seq model (linear SVM) with the WSI model (Resnet18) were combined by multiplying the probability scores of each single-modality model. Model performance was measured with the F1 metric.

For cancer type prediction, the multimodality model achieved an F1 score of 0.95 on the test set. About 40% of the cancer types benefited from a synergistic effect by combining the two modalities. Cancer types and percent increase in F1 scores, respectively, that benefit most by combining modalities are: Cervical squamous cell carcinoma and endocervical adenocarcinoma (4.23%), Cholangiosarcoma (6.66%) and Uterine carcinosarcoma (4%). Interestingly, in other cancer types the combination did not result in improved predictive scores compared to a single modality model, e.g. in Rectum adenocarcinoma, Sarcoma or Stomach adenocarcinoma. For Prostate cancer grading, Gleason score prediction of patterns 3/4/5, combined multi-modality model earned 0.73 F1 outperforming the single modality models.

By combining histopathology imaging and omics modalities it has been demonstrated that there are synergistic effects in predictive power for both cancer-related research questions. There was an improved predictive performance in 40% of the classified cancer types by taking both modalities. Imaging or omics modalities alone can be sufficient in some cases and their strengths are very problem-specific.

### Example 1: AIMM-Multi Modal H&E+RNA Predictions for Cancer Patients

The prediction of multiple targets (like the type of the cancer or the gender of the patient) was used for cancer patients, in two modalities, namely H&E Whole Slide Images and RNA sequence data.

The goal was to determine if it possible to predict these targets from the patient data and what target is best predicted by which modality. In addition it was a goal to determine if it was possible to create stronger predictions by combining both modalities, and which targets should be used for predicting the cancer type, the gender, and the Gleason score of the patient. It has been shown how the modalities can be combined in two ways: —by taking the individual models of the different modalities and then processing and combining their predictions, or by combining the data of the modalities to create a single model that fuses the data.

Models trained on H&E and on RNA data will exploit different information. Therefore when they are wrong, the errors originate from different analysis, i.e. one or more of the models are not correctly correlated. In the present invention we show that we can use this to add an option of “rejecting” slides where both modalities disagree, achieving a much higher accuracy with the remaining slides.

In practice when using Deep Learning models it is highly desirable to know when not to use the model decision. Deep Learning models can in some cases be overconfident, but wrong. In the case of a clinical trial for example, if a risk model thinks the patient is high risk, but with a very wide confidence interval, or very high uncertainty, it would be safest to reject the data point and not apply the model. In other words, if it was possible to identify in advance data where the models are prone to fail, it would be best not to use the models there, and perhaps revert to something else (like a human in the loop).

However, achieving this, and quantifying how much deep neural networks are uncertain, can be difficult, and is an active research question. There is a trade-off between how much data is rejected (the lower the better) and how accurate the model becomes on the remaining group. It is demonstrated herein that two uncorrelated modalities that use different features are a very reliable way to achieve this with very good results.

This work was conducted on a very large scale of slides: 10,000 slides, with data stored and processed in IRISE. The key results are that for Cancer Type prediction for 30 categories, the multi-modality model achieves 95% F1 score on a large and diverse test set. Cancer types that benefit most by combining modalities are: CESC (4.23%), CHIOL (6.66%) and UCS (4%), while the cancer type for which the combination did not improve compared to a single model are: Read (−3.13%), SARC (−2.31%) and STAD (−1.28%).

Some of the cancer types are highly predictive using a single modality. For the Genomic modality the most predictive cancer types are; BARC (100% F1), DLBC (100% F1), GBM (100% F1), KIRP (100% F1), LGG (100% F1), OV (100% F1), PAAD (100% F1), PRAD (100% F1), TGCT (100% F1), THYM (100% F1) and UVM (100% F1). For the Imaging modality the most predictive cancer type is UVM (100% F1).

By rejecting slides that disagree from both modalities, there is a 98% F1 score (with the price of rejecting 16% of the cases). By rejecting slides that disagree from both modalities for Gleason Pattern Prediction of patterns 3 and 4, there is a 100% F1 score (with the price of rejecting 39% of the cases), or 87% with the imaging modality alone applied on all cases.

For Gleason Pattern Prediction of patterns 3/4/5, combined multi modality model gets 73% F1, and by rejecting slides where the modalities disagree there is a 90% F1 score (with the price of rejecting 50% of the cases). This is a very high accuracy considering no supervised annotations are used.

We also report results for predicting Luad/Lusc overall survival risk prediction (62%+CINDEX) and LUAD/LUSC classification (95% AUC).

For prediction of gender, the imaging model is unable to predict the gender (with AUCs in the range 50-60), while the RNA sequences are highly accurate. This shows that for some prediction targets it does not make sense to use some modalities.

### Genomic Stream

### Data Sets

Public available gene expression data from TCGA (https://portal.gdc.cancer.gov/) has been used. Only samples from 32 primary tumor sites were selected. We downloaded Gene expression data in fragment per kilobase million (FPKM) with 56,602 Ensemble gene identifiers. In order to allow for a better comparison between other data sources (e.g. GTEX), we normalized the FPKM data into TPM (Transcripts per million) using following equation:

$$TPM = \frac{FPKMi}{\sum FKP} \times {10}^{6}$$

### Data Preparation

Only protein-encoding genes were included and the genes were selected via API from Ensembl (https://rest.ensembl.org/documentation/info/lookup). Several experiments were conducted to see which dataset achieves best accuracy. Excluded genes containing zero expression values and considering only protein-encoding genes gave best results and led to a final gene set of 10125 selected genes. The final dataset was split into a predefined training (6614 samples) and validation (1674 samples) component.

TABLE 1
Results of different conducted experiments with different gene sets.

| | Number of | |
|---|---|---|
| Experiment | Genes | Accuracies |
| FPKM with all | 56612 | Accuracy: 94.23% |
| genes | | Balanced Accuracy |
| | | 90.66% |
| | | F1 Score: 0.91 |
| FPKM without | 10125 | Accuracy: 96.06% |
| zero expression | | Balanced Accuracy |
| | | 94.85% |
| | | F1 Score: 0.95 |
| FPKM only protein | 19552 | Accuracy: 94.02% |
| encoding genes | | Balanced Accuracy |
| | | 91.43% |
| | | F1 Score: 0.92 |
| FPKM only protein | 10135 | Accuracy: 96.06% |
| encoding genes | | Balanced Accuracy 94% |
| without zero | | F1 Score: 0.94 |
| gene expression | | |

Table 1 shows results of a first experiment to determine which gene expression data set shows better performance in predicting cancer types. Reducing the full data set to only protein encoding genes and removing genes with zero expression led to the highest accuracy.

### Preprocessing, Feature Selection, ML Design and Model Explainability

To develop and optimize the best model for each specific use case, a manual approach and an automated approach were tested. The manual approach includes normalisation, label encoding, feature reduction and hyperparameter tuning steps to find the best setting for the final Machine Learning model. We considered an XGB classifier as this algorithm is not defined in the auto-sklearn tool. To find the best set of features we first performed a Lasso regularisation followed by a recursive feature elimination (RFE) step.

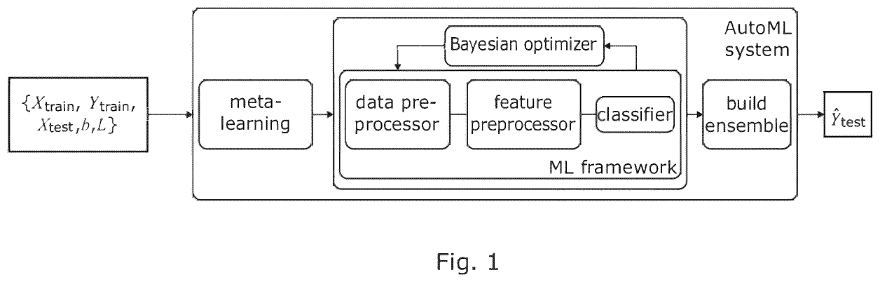

For the automated approach we used the auto-sklearn library in Python, which allows the user a fast and easy implementation of Machine Learning experiments with all necessary steps such as preprocessing, feature and model selection plus hyperparameter tuning. The library contains 16 different machine learning models and 18 different feature selection methods. As a quality metric we used F1 macro score. The auto-sklearn pipeline is shown in FIG. 1.

Machine learning models are thought to have better performance compared to simpler models, but at the cost of losing explainability and intelligibility. The SHAP (SHapley Additive explanations) algorithm, developed by Lundberg and Lee in 2017, is the state-of-the-art tool in Machine Learning to better interpret and inverse engineer the output of any predictive algorithm.

### Results

### Gender Prediction

For predicting gender a total of 194 models were trained using the auto-sklearn pipeline and 45 different XGB models using the training set and a 5-fold stratified cross-validation approach which preserves the percentage of samples for each class.

TABLE 2
Test score of both models for various selected quality metrics

| Score | SVM (AutoML) Test | XGB Test |
|---|---|---|
| Accuracy | 0.94 | 0.92 |
| Balanced Accuracy | 0.94 | 0.92 |
| F1 (Macro) | 0.94 | 0.92 |
| MCC | 0.87 | 0.84 |

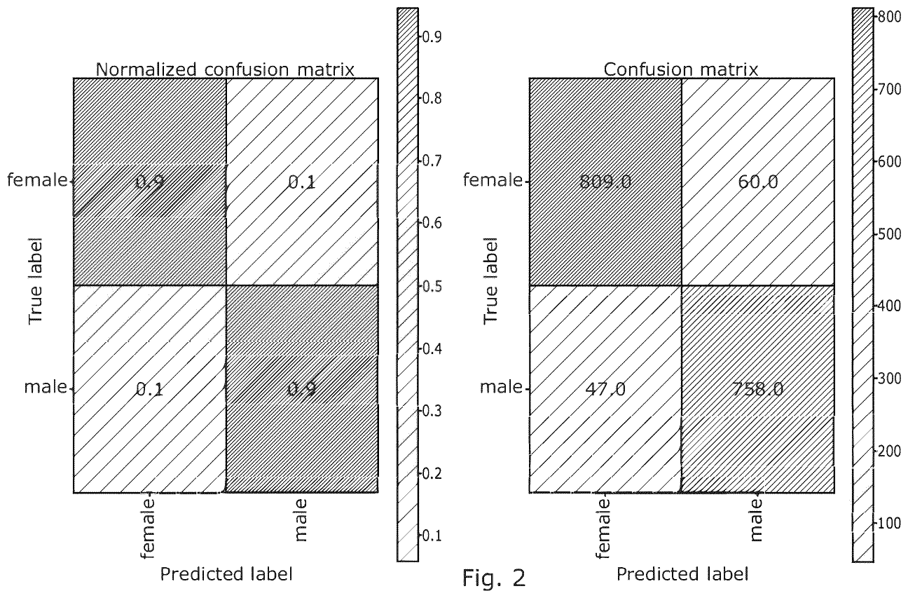

As best model, a SVM model was determined with a F1 score of 0.94. The processing and SVM classification pipeline for gender prediction was class-balancing of the input, followed by L1 (Lasso) feature reduction, then linear SVM and finally output. Further, a confidence interval was computed for the best model (SVM) using a bootstrapping approach with 1000 boots. The 95% confidence interval ranged from 0.94 to 0.96 for the F1 score. The achieved test score of 0.94 falls into the computed confidence interval and indicates a representative sample selection of the test set. The confusion matrix is shown in FIG. 2.

The class wise accuracies on cancer level are summarised in Table 3. The following have the lowest F1 score: Cervical squamous cell carcinoma (cesc), Prostate adenocarcinoma (prad), Testicular Germ Cell Tumors (tgct), Uterine Carcinosarcoma (ucs), and Uterine Corpus Endometrial Carcinoma (ucec).

TABLE 3
Class wise results for the Linear SVM classifier. In
red highlighted the classes with the lowest F1-score

| Class | | | | Sample |
|---|---|---|---|---|
| Cancer Type | Precision | Recall | F1 score | Number |
| acc | 0.85 | 0.81 | 0.79 | 25 |
| blca | 0.92 | 0.92 | 0.92 | 87 |
| brca | 0.78 | 0.99 | 0.85 | 228 |
| cesc | 0.50 | 0.49 | 0.49 | 42 |
| chol | 1.00 | 1.00 | 1.00 | 7 |
| coad | 0.93 | 0.93 | 0.93 | 111 |
| dlbc | 1.00 | 1.00 | 1.00 | 8 |
| esca | 0.75 | 0.96 | 0.81 | 27 |
| gbm | 0.95 | 0.85 | 0.89 | 27 |
| hnsc | 0.91 | 0.92 | 0.91 | 73 |
| kirp | 0.88 | 0.88 | 0.88 | 37 |
| 1gg | 0.97 | 0.96 | 0.97 | 122 |
| lihc | 0.87 | 0.90 | 0.89 | 74 |
| luad | 0.94 | 0.94 | 0.94 | 90 |
| lusc | 0.83 | 0.89 | 0.83 | 51 |
| meso | 0.83 | 0.89 | 0.83 | 13 |
| OV | 1.00 | 1.00 | 1.00 | 14 |
| paad | 0.98 | 0.96 | 0.94 | 41 |
| pcpg | 0.96 | 0.93 | 0.94 | 37 |
| prad | 0.50 | 0.48 | 0.49 | 77 |
| read | 0.90 | 0.95 | 0.92 | 27 |
| sarc | 0.84 | 0.83 | 0.83 | 55 |
| skcm | 0.86 | 0.86 | 0.90 | 59 |
| stad | 0.91 | 0.89 | 0.89 | 73 |
| tgct | 0.50 | 0.48 | 0.49 | 32 |
| thca | 0.99 | 0.92 | 0.99 | 104 |
| thym | 0.95 | 0.97 | 0.96 | 26 |
| uvec | 0.50 | 0.47 | 0.48 | 72 |
| ucs | 0.50 | 0.46 | 0.48 | 13 |
| uvm | 0.73 | 0.71 | 0.71 | 14 |

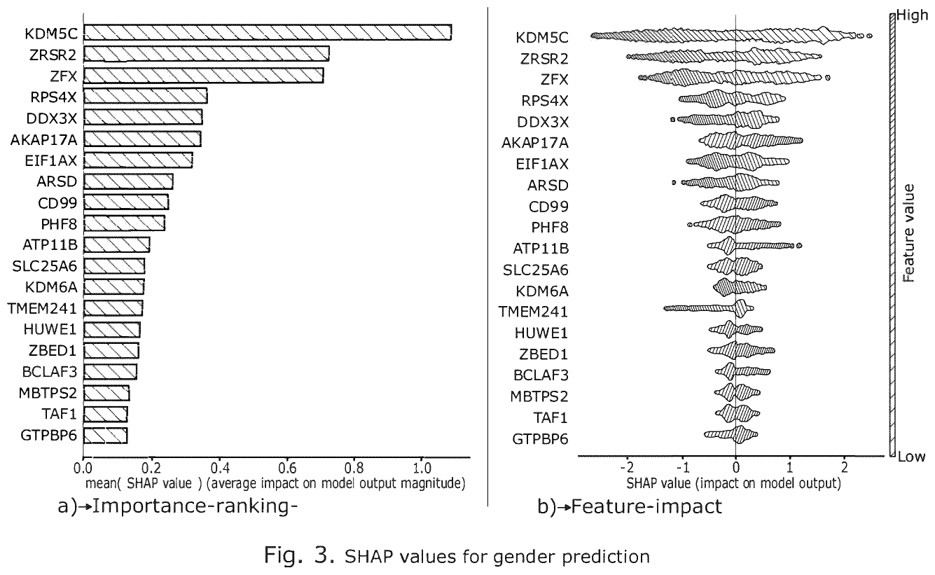

The SHAP values for gender prediction are shown in FIG. 3.

### Cancer Type Prediction

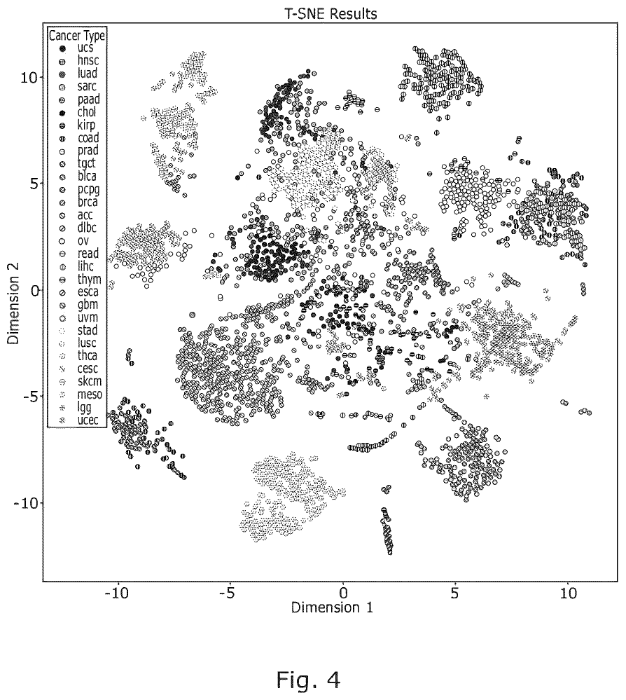

A T-distributed Stochastic Neighbor Embedding (t-SNE) visualisation revealed the high potential of using the selected data sources to classify cancer types as shown in (FIG. 4).

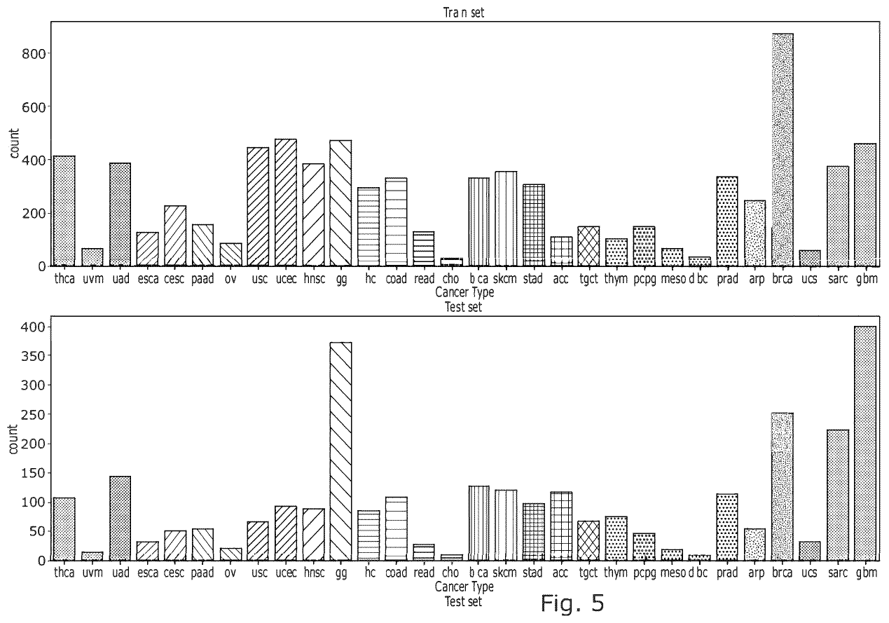

For predicting gender a total of 145 models were trained using the auto-sklearn pipeline and 45 different XGB models using the training set and a 5-fold stratified cross-validation approach which preserves the percentage of samples for each class. The class distribution of the training and test sets are shown in FIG. 5.

TABLE 4
Test score of both models for various selected quality metrics

| Score | LDA (AutoML) Test | XGB Test |
|---|---|---|
| Accuracy | 0.96 | 0.93 |
| Balanced Accuracy | 0.94 | 0.89 |
| F1 (Macro) | 0.95 | 0.90 |
| MCC | 0.96 | 0.88 |

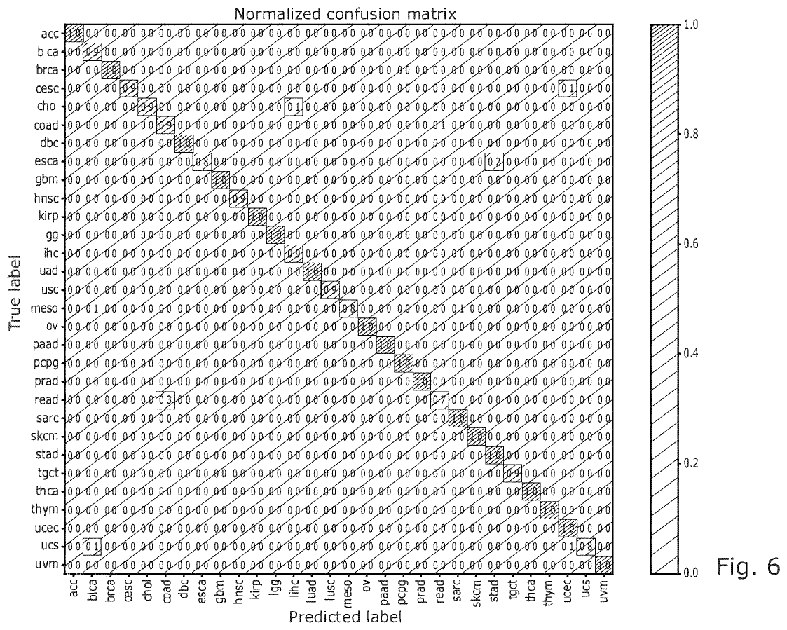

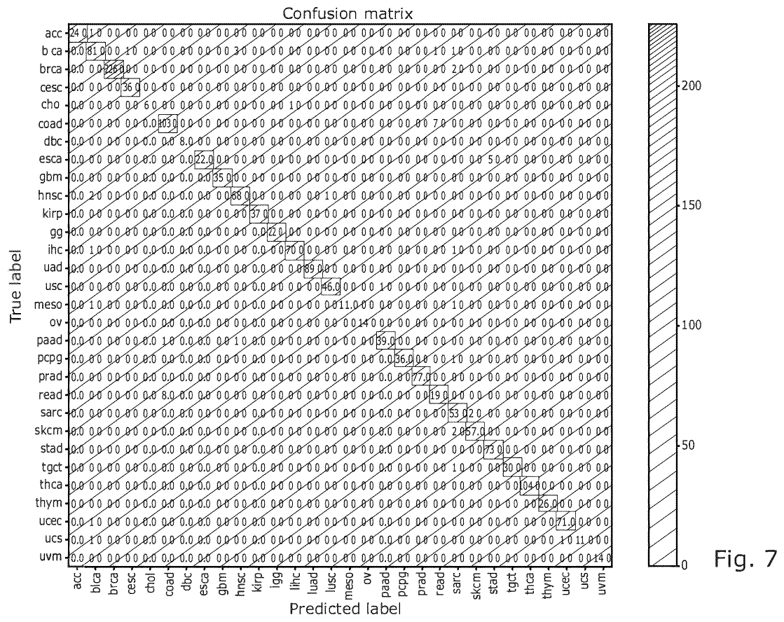

As best models a LDA model was determined with a F1 score of 0.94 outperforming the XGB classifier. The processing and LDA classification of the Auto-sklearn pipeline for gender prediction was select rates classification of the input, followed by LDA and then output. Further, a confidence interval was computed for the best model (LDA) using a bootstrapping approach with 1000 boots. The 95% confidence interval ranges from 0.93 to 0.96. The achieved test score of 0.94 falls into the computed confidence interval and indicates a representative sample selection of the test set. The confusion matrices are shown in FIGS. 6 and 7.

Based on the class wise accuracies on cancer level summarized in Table 3, the following have the lowest F1 score: Cervical squamous cell carcinoma (cesc), Prostate adenocarcinoma (prad), Testicular Germ Cell Tumors (tgct), Uterine Carcinosarcoma (ucs), and Uterine Corpus Endometrial Carcinoma (ucec).

TABLE 5
Class wise results for the Linear SVM classifier.

| Class | | | | Sample |
|---|---|---|---|---|
| Cancer type | Precision | Recall | F1 score | Number |
| Acc | 1.00 | 0.96 | 0.98 | 25 |
| Blca | 0.99 | 0.92 | 0.95 | 87 |
| Brca | 1.00 | 0.99 | 1.00 | 228 |
| Cesc | 0.90 | 0.86 | 0.88 | 42 |
| Chol | 0.75 | 0.86 | 0.80 | 7 |
| Coad | 0.90 | 0.88 | 0.89 | 111 |
| Dlbc | 1.00 | 1.00 | 1.00 | 8 |
| Esca | 0.92 | 0.81 | 0.86 | 27 |
| Gbm | 1.00 | 1.00 | 1.00 | 35 |
| Hnsc | 0.95 | 0.95 | 0.95 | 73 |
| Kirp | 1.00 | 1.00 | 1.00 | 37 |
| Lgg | 1.00 | 1.00 | 1.00 | 122 |
| Lihc | 0.99 | 0.96 | 0.97 | 74 |
| Luad | 0.98 | 0.96 | 0.97 | 90 |
| Lusc | 0.88 | 0.96 | 0.92 | 51 |
| Meso | 1.00 | 0.92 | 0.96 | 13 |
| Ov | 1.00 | 1.00 | 1.00 | 14 |
| Paad | 1.00 | 1.00 | 1.00 | 41 |
| Pcpg | 1.00 | 0.97 | 0.99 | 37 |
| Prad | 1.00 | 1.00 | 1.00 | 77 |
| Read | 0.57 | 0.63 | 0.60 | 27 |
| Sarc | 0.90 | 0.96 | 0.93 | 55 |
| Skcm | 0.97 | 0.97 | 0.97 | 59 |
| Stad | 0.93 | 0.97 | 0.97 | 73 |
| Tgct | 1.00 | 1.00 | 1.00 | 32 |
| Thca | 1.00 | 1.00 | 1.00 | 104 |
| Thym | 1.00 | 1.00 | 1.00 | 26 |
| Uvec | 0.91 | 1.00 | 0.95 | 72 |
| Ucs | 1.00 | 0.92 | 0.96 | 13 |
| Uvm | 1.00 | 1.00 | 1.00 | 14 |

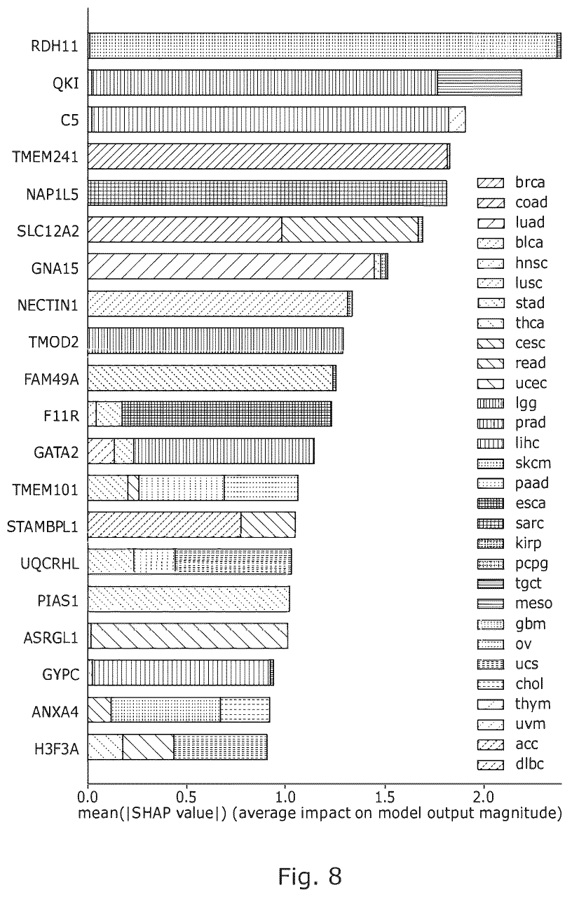

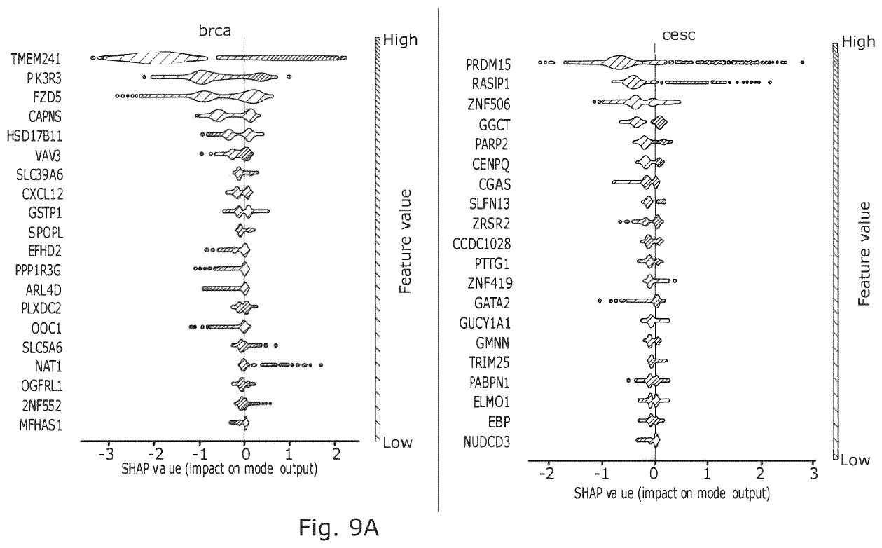

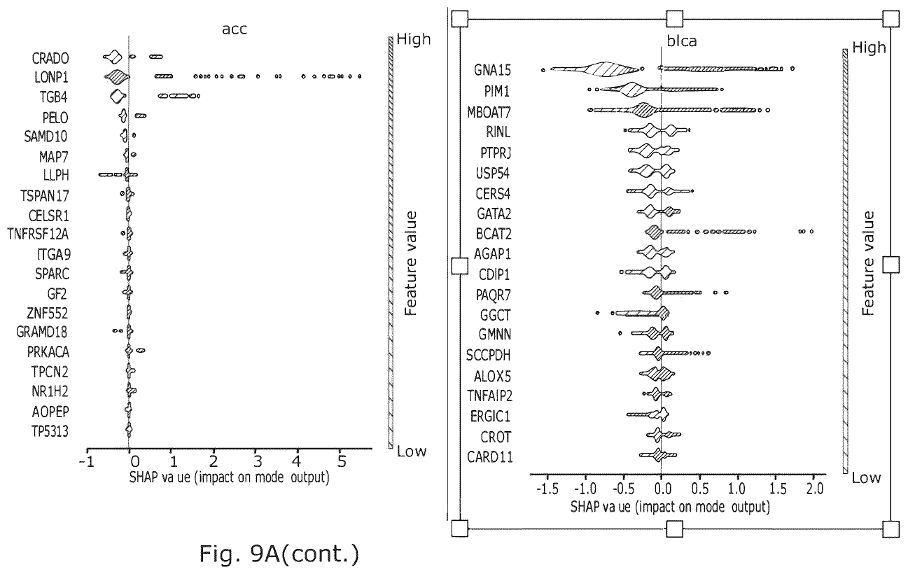

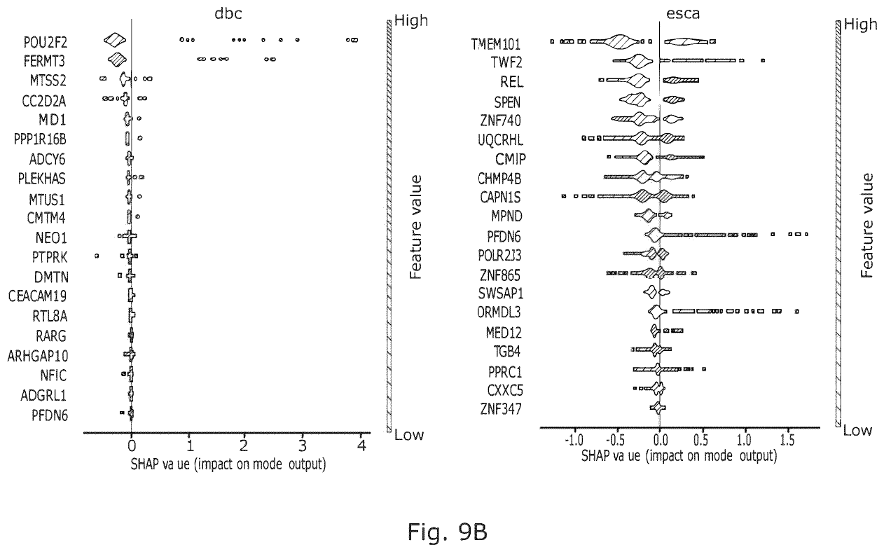

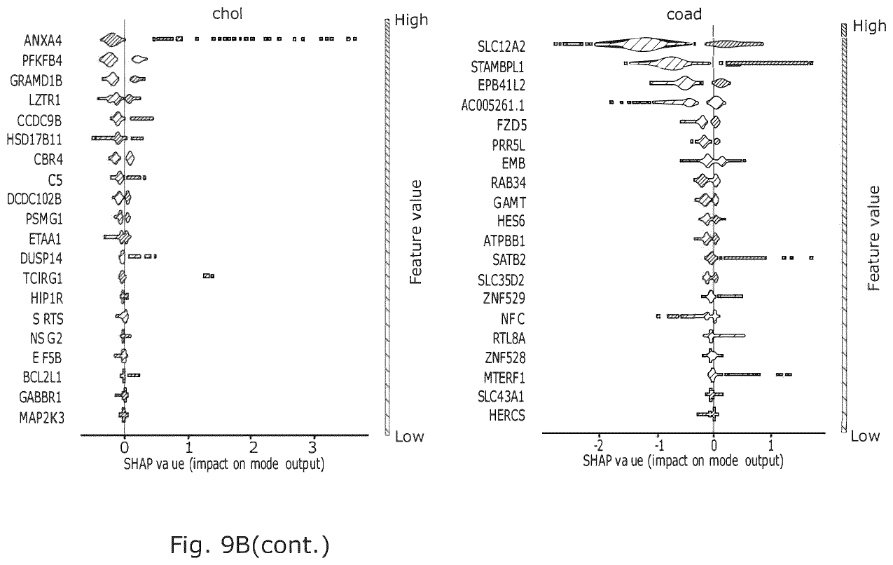

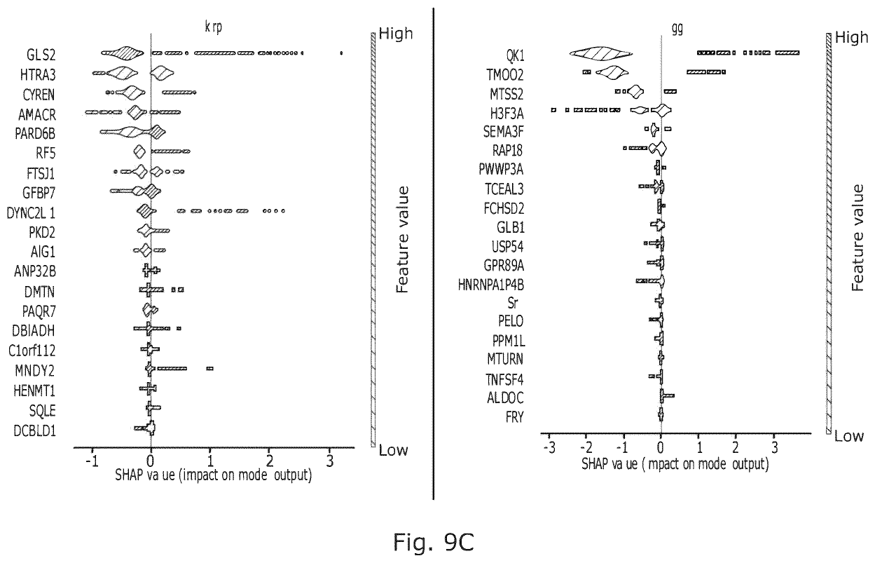

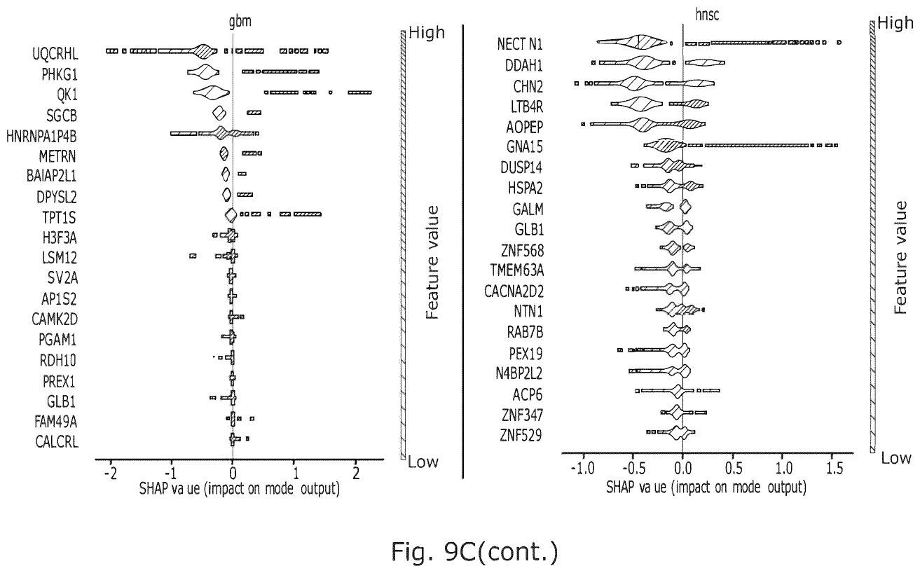

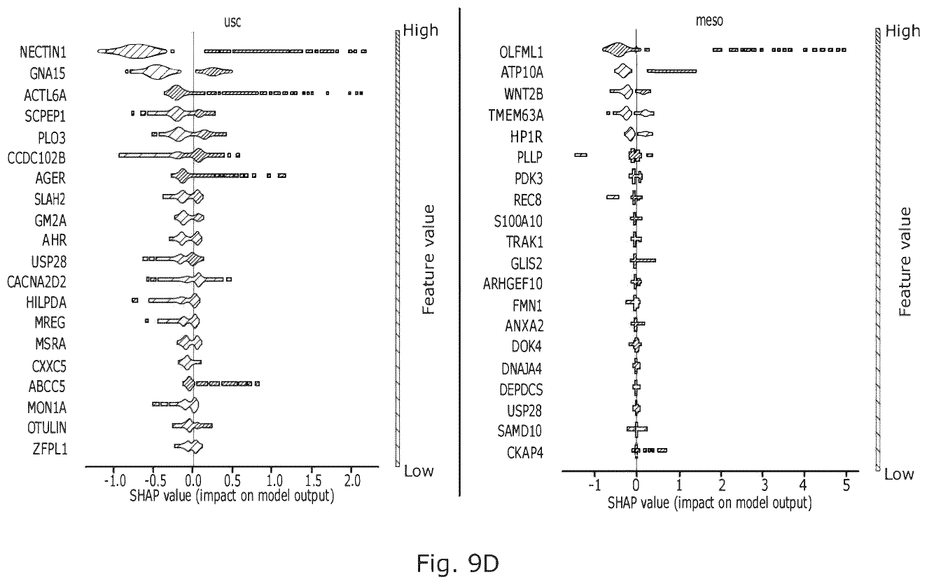

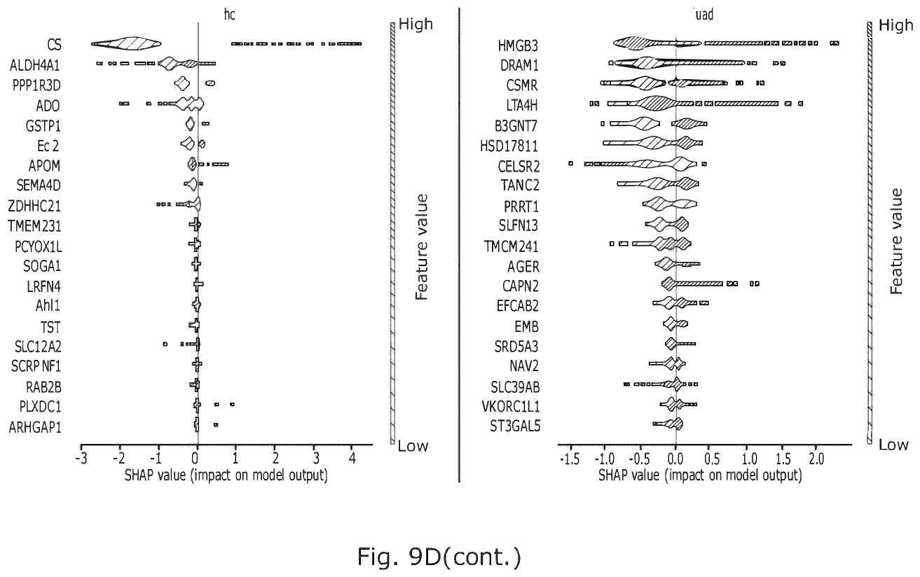

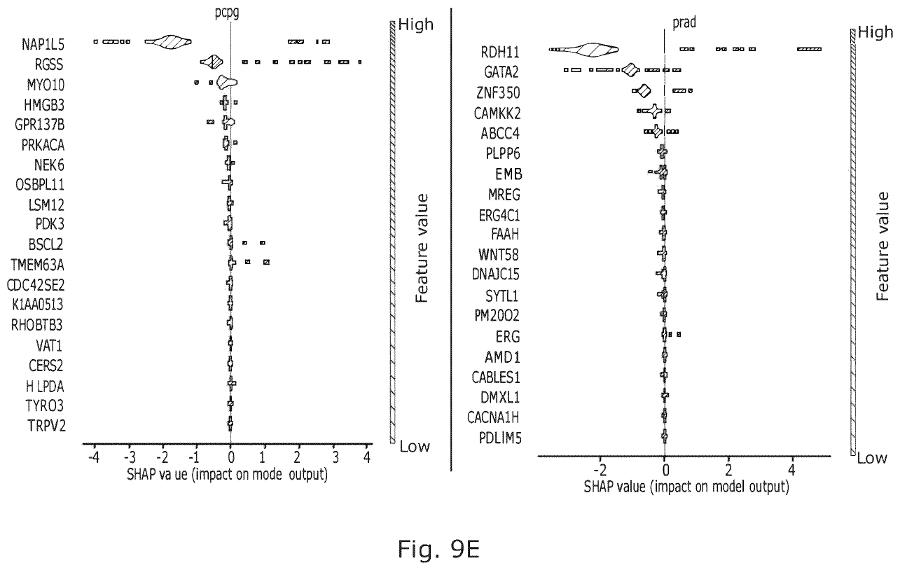

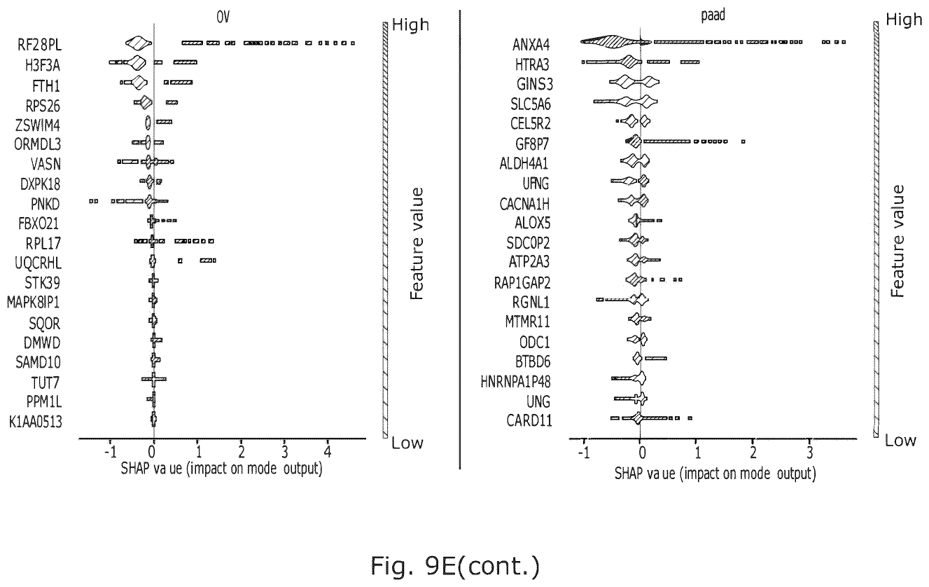

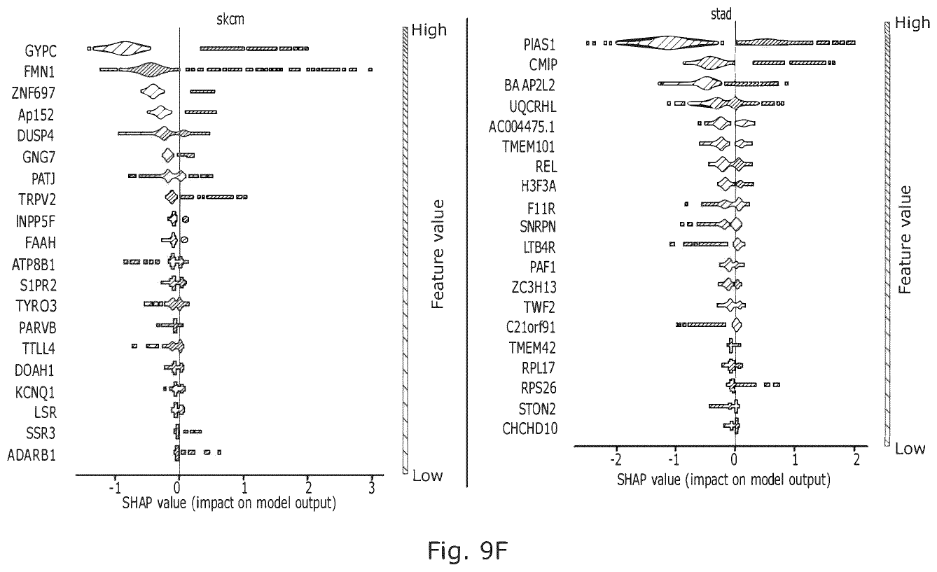

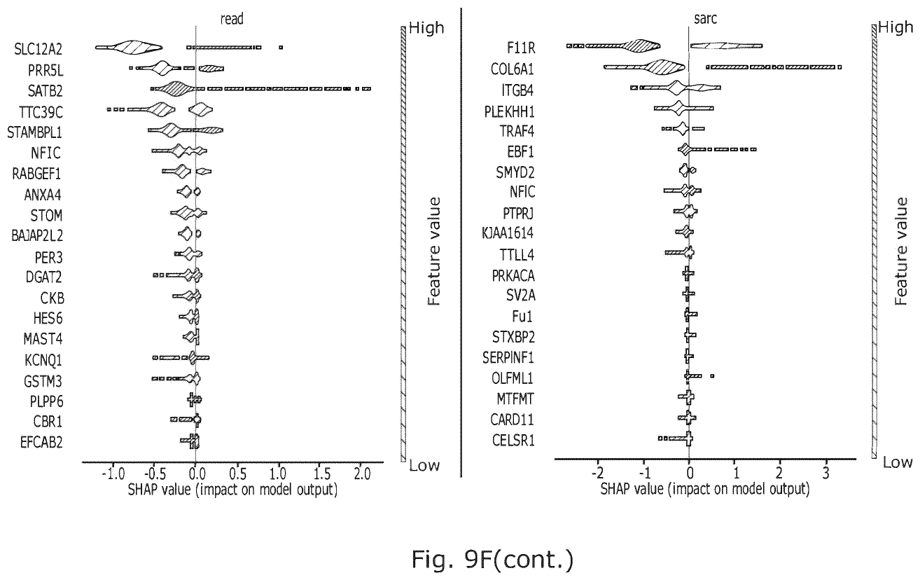

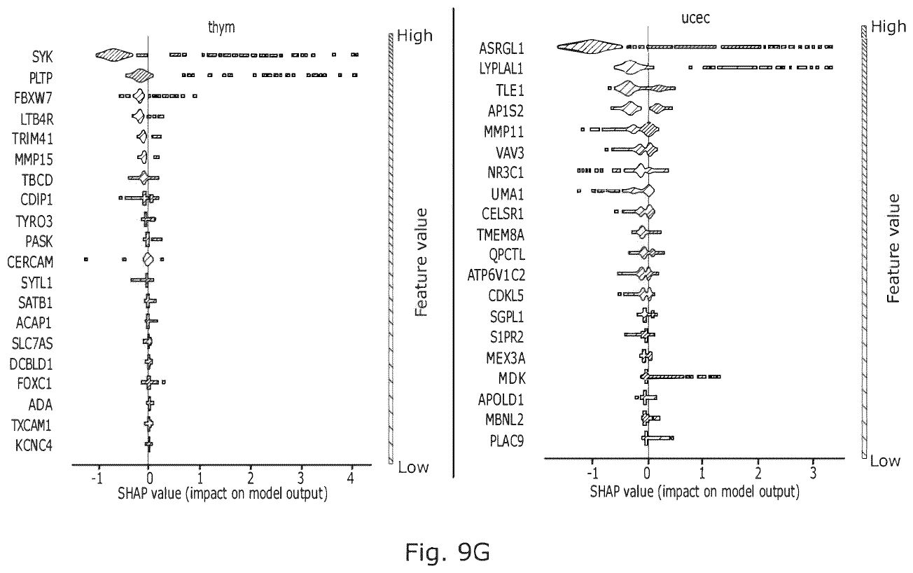

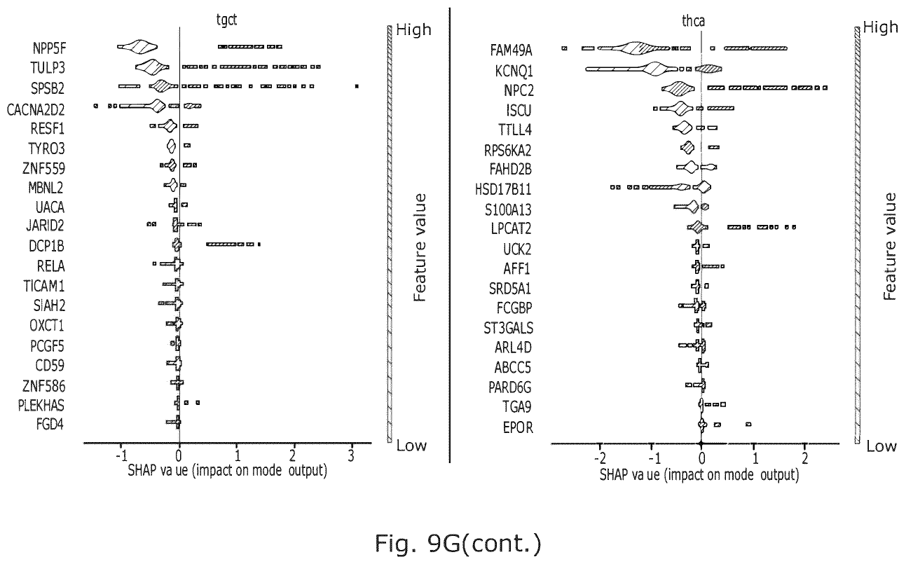

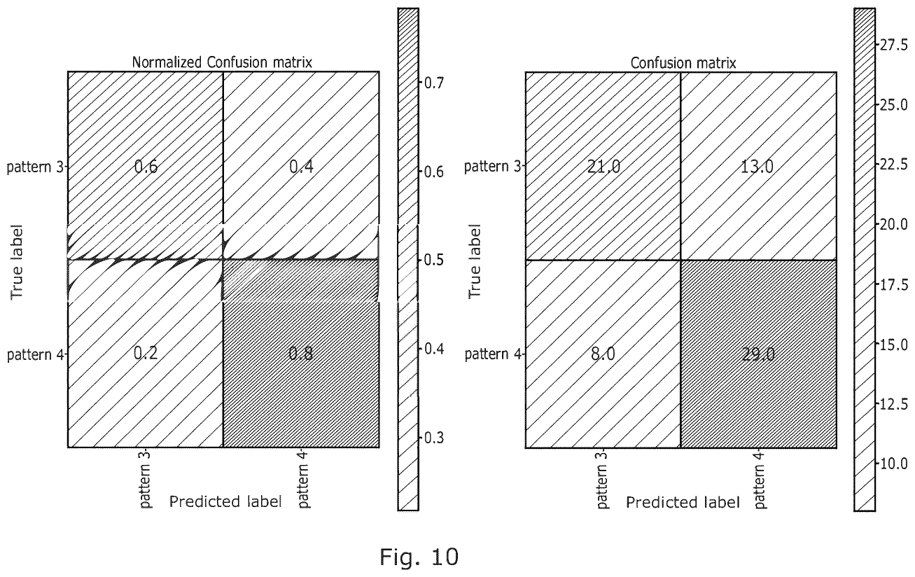

FIG. 8 shows the SHAP values for cancer type prediction based on specific genes and FIGS. 9A-9G show the feature impact for each cancer type in the prediction.

### Gleason Score Prediction

Gleason score is a grading system to determine the aggressiveness of prostate cancer. The score ranges from 1 to 5 and describes how much the potentially cancerous tissue from a biopsy looks like healthy tissue. The majority of the cancer has grade 3 or higher.

For predicting gender we trained models using the auto-sklearn pipeline and XGB algorithm using the training set and a 5-fold stratified cross-validation approach which preserves the percentage of samples for each class.

### Primary Gleason Score

Only three of the Gleason classes (Gleason 3, 4 and 5) were examined and several models were developed: namely Gleason 3 vs. Gleason 4 and Gleason 3 vs. Gleason 4 vs. Gleason 5. For Gleason 5 there were a limited number of samples available.

Gleason 3 vs. Gleason 4

For Gleason 3 vs. 4 the full feature set for the auto-sklearn pipeline was used. In total, 606 different algorithms and 65 XGB models with different parameter settings were tested resulting in a F1 test score of 0.74. As best model a linear SGD (Stochastic Gradient Descent) was chosen. The Auto-sklearn pipeline for Gleason prediction was class balancing of the input, followed by selection of the percentile, then SGD and output.

TABLE 6
Test score of both models for various selected quality metrics

| Score | LDA (AutoML) Test | XGB Test |
|---|---|---|
| Accuracy | 0.74 | 0.71 |
| Balanced Accuracy | 0.71 | 0.70 |
| F1 (Macro) | 0.71 | 0.70 |
| MCC | 0.48 | 0.44 |

Gleason 3 vs. Gleason 4 vs Gleason 5

For Gleason 3 vs. 4 vs. 5 we used the full feature set for the auto-sklearn pipeline and the XGB algorithm. In total, 510 different algorithms and 65 XGB models with different parameters settings were tested resulting in a F1 test score of 0.68. As the best model a SGD algorithm was chosen. The Auto-sklearn pipeline for cancer type prediction was L1 regularisation of the input followed by SGD and then the output.

TABLE 7
Test score of both models for various selected quality metrics

| Score | LDA (AutoML) Test | XGB Test |
|---|---|---|
| Accuracy | 0.68 | 0.62 |
| Balanced Accuracy | 0.62 | 0.54 |
| F1 (Macro) | 0.64 | 0.58 |
| MCC | 0.42 | 0.32 |

Further, a confidence interval was computed for the best model (SGD) using a bootstrapping approach with 1000 boots. The 95% confidence interval ranges from 0.55 to 0.76.

The achieved test score of 0.64 falls into the computed confidence interval and indicates a representative sample selection of the test set.

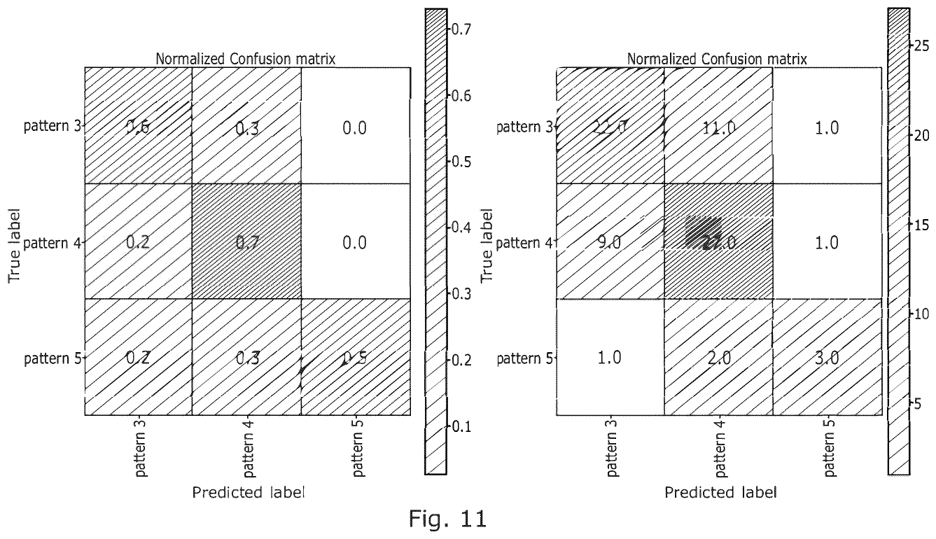

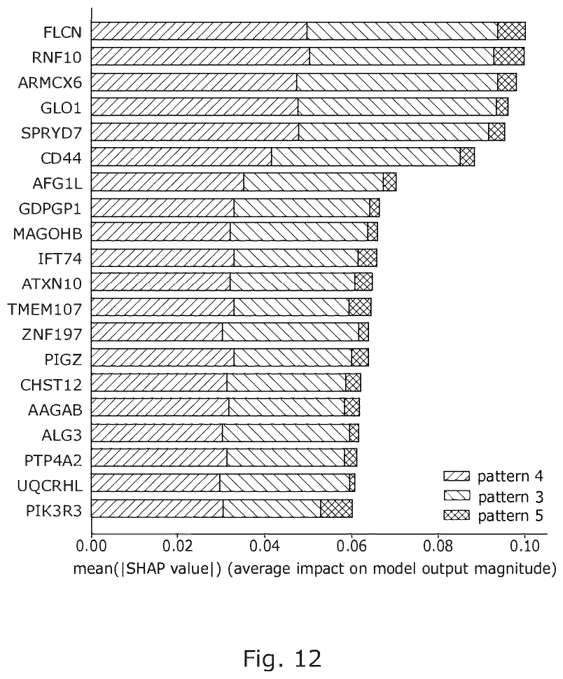

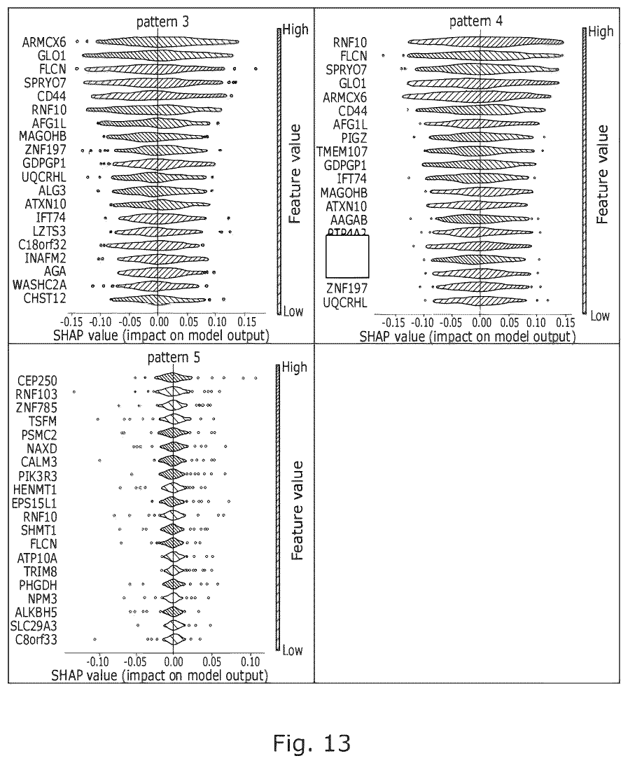

FIG. 11 shows the confusion matrix for gender prediction and FIG. 12 shows the SHAP values for primary Gleason Score prediction, whilst FIG. 13 shows the feature impact for each Gleason pattern in the prediction imaging stream.

Imaging Stream: Predicting Endpoints from the TCGA LUAD/LUSC H&E Slides

### Data Preparation

The IRISE study had 4554 sample slides. A tumor detection algorithm originally developed for DLBCL was applied on the slides, as an approximation of filtering out non cellular content. It was inspected visually to verify the mask makes sense. Then an Image-Net pretrained Resnet50 model was applied on 256×256 tiles from the cellular regions, and 2048 features were extracted per tile from the penultimate layer.20% of the slides, randomly selected, were reserved as a test set. This was done for several image magnification factors: 5×, 10×, 20×, 40×.

### Script for Creating Embeddings in 10× Magnification for LUSC:

sbatch --mem 300gb --array 0-7 --partition gpu --gres=gpu:1
scripts/embeddings/create_embeddings_wsi.py --slides_dir
/pstore/data/dspta/data/aimm/4554_lusc/4554/slides --backbone_name resnet50
--output_dir
/pstore/data/dspta/data/aimm/4554_lusc/resnet50_imagenet_embeddings_10x
--batch_size 128 --filter_type filter_based_on_qc_and_tumor_detection --study_id
4554 --experiment_id 5002 --magnification 10 --extrapolation_tolerance 999
--iris_url=https://iris-e-explorer.navify.com --analysis_masks_dir
/pstore/data/dspta/data/aimm/4554_lusc/4554/analysis_masks
Creating Embeddings in 10x magnification for LUAD:
sbatch --mem 300gb --array 0-7 --partition gpu --gres=gpu:1
scripts/embeddings/create_embeddings_wsi.py --slides_dir
/pstore/data/dspta/data/aimm/4554_luad/4554/slides --backbone_name resnet50
--output_dir
/pstore/data/dspta/data/aimm/4554_luad/resnet50_imagenet_embeddings_10x
--batch_size 128 --filter_type filter_based_on_qc_and_tumor_detection --study_id
4554 --experiment_id 5002 --magnification 10 --extrapolation_tolerance 999
--iris_url=https://iris-e-explorer.navify.com --analysis_masks_dir
/pstore/data/dspta/data/aimm/4554_luad/4554/analysis_masks

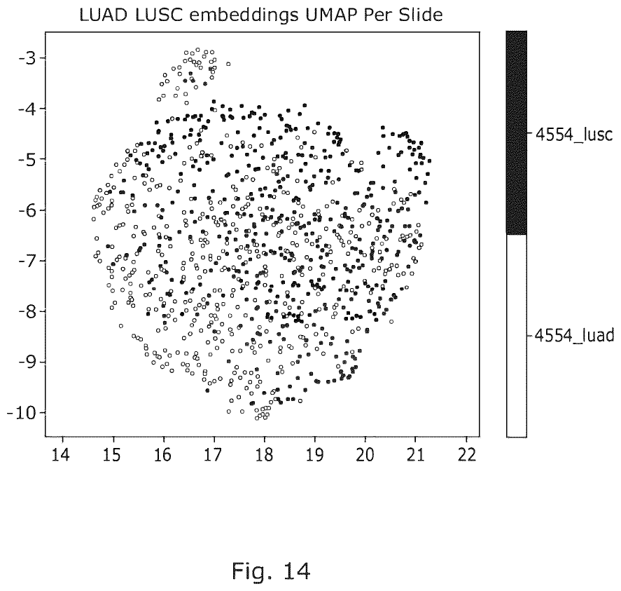

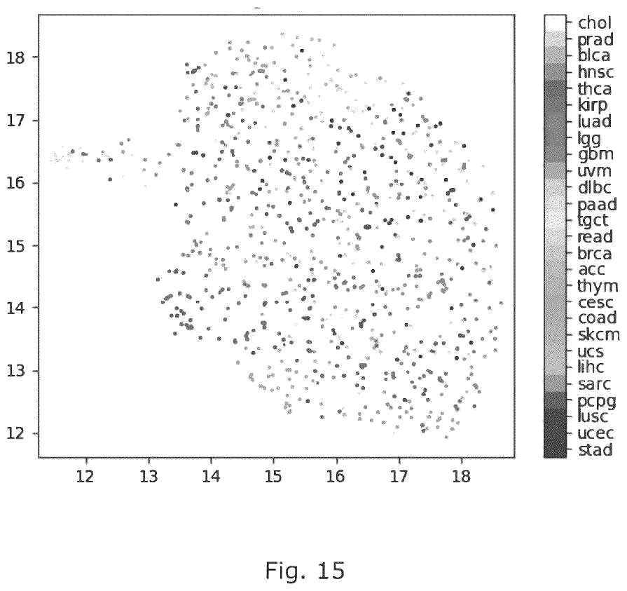

Exploratory Data Analysis was performed by clustering the created 10× embeddings with the UMAP algorithm, to show that there is some difference between how the LUAD and LUSC slides look. The LUAD/LUSC visualisation of the embeddings is shown in FIG. 14.

Note: the visualization was based on modifying this script:

scripts/general/umap_per_study.py
Branch: release/phase1b
(And modifying the plot title inside the code)
Run command: python scripts/general/umap_per_study.py <json file with list of embedding files
per slide>

Endpoint: Risk (based on number of days for overall survival).

This model was trained using Attention-MIL on the embeddings, with a Cox Regression loss function, using the number of days to event (this essentially learns to rank the risks of different patients based on their number of days to event), giving the following results:

TABLE 8

| Magnification | LUAD | LUSC | LUAD + LUSC |
|---|---|---|---|
| 10x | Cindex: 66% | Cindex: 61% | Cindex: 62% |
| 20x | Cindex: 62% | Cindex: 59% | Cindex: 62% |

Model training occurred with IRISAI (an open source digital pathology imaging platform available through Github and Pyris large language model (LLM) microservice) using the following scripts:

sbatch --mem 150gb --error /pstore/data/dspta/data/aimm/logs/slurm.%j.err --output
/pstore/data/dspta/data/multimodal_luad_lusc/logs/slurm.%j.out --partition gpu --gres gpu:4
scripts/weakly_supervised/trainers/train_mil_pfs.py --epochs 400 --lr 0.001 --embeddings
“/pstore/data/dspta/data/aimm/jsons/luad_lusc_20x.json” --testing_frequency 1 --labels_cols_list
num_of_days
event --labels_file_path
/pstore/data/dspta/data/multimodal_luad_lusc/metadata/4554_pfs_luad_lusc.csv
--output_path /pstore/data/dspta/data/aimm/checkpoints --batch_size 128 --algorithm
attention_mil_pfs
--bag_sample 256 --dropout 0.75 --kl_loss_weight 0.5 --percent_train_data 0.8 --metric AUC --
seed 2
--weight_decay 0.001 --low_risk_threshold 730 --test_slides_path
/pstore/data/dspta/data/aimm/splits/luad_lusc_test.txt

Endpoint: LUAD/LUSC cancer subtype classification.

The result is shown in Table 9.

TABLE 9

| Magnification | Epoch | AUC |
|---|---|---|
| 10x | | 93% |
| 20x | | 95% |

Model training occurred utilizing the IRISAI open source tool with the following script/parameters:

sbatch --mem 300gb --error /pstore/data/dspta/data/aimm/logs/sturm.%j.err --output
/pstore/data/dspta/data/aimm/logs/slurm.%j.out --partition gpu --gres gpu:4
scripts/weakly_supervised/trainers/train_mil.py --epochs 400 --testing_frequency 1 --
labels_cols_list “Cancer
Type” --labels_file_path /pstore/data/dspta/data/aimm/metadata/4934_all.csv --output_path
/pstore/data/dspta/data/aimm/checkpoints --batch_size 256 --algorithm attention_mil --
bag_sample 256 --dropout
0.75 --kl_loss_weight 5 --classes_names ‘luad:0,lusc:1’ --metric AUC --weight_decay 0.000 --lr
0.001
--embeddings /pstore/data/dspta/data/aimm/4554/resnet50_imagenet_embeddings_10x/ --seed 0
--test_slides_path /pstore/data/dspta/data/aimm/splits/4934_resnet18_10x_cancer_type_test.txt -
-percent_train_data 0.8

### Endpoint: LUAD/LUSC Gender Prediction

### Result: 67% AUC

Model training occurred utilizing the IRISAI open source tool with the following script/parameters:

sbatch --mem 200gb --error /pstore/data/dspta/data/aimm/logs/slurm.%j.err --output
/pstore/data/dspta/data/aimm/logs/slurm.oj.out --partition gpu --gres gpu:4
scripts/weakly_supervised/trainers/train_mil.py --epochs 400 --testing frequency 1 --
labels_cols_list gender
--labels_file_path /pstore/data/dspta/data/aimm/metadata/4934_gender.csv --output_path
/pstore/data/dspta/data/aimm/checkpoints --batch_size 256 --algorithm attention_mil --
bag_sample 256 --dropout
0.75 --kl_loss_weight 5 --classes_names ‘female:0,male:1’ --metric AUC --weight_decay 0.000 --
lr 0.001
--embeddings /pstore/data/dspta/data/aimm/jsons/4934_resnet18_10x.json --seed 0 --
test_slides_path
/pstore/data/dspta/data/aimm/splits/4934_10x_test.txt --percent_train_data 0.8

Predicting Endpoints from the Full TCGA H&E Dataset with ˜10,000 Slides

### Dataset Preparation

To handle the scale of slides, batches of slides were downloaded and then extracted with ImageNet-Pretrained Resnet18 embeddings from them as before on 256×256 tiles. Unlike previously, tumor detection masks were not used, and instead an IRISAI image processing based ‘ATD—Automatic Tissue Detection’ filter was used to extract embeddings only from tiles in tissue regions. Unlike before, Resnet18 was used and not Resnet50, so the features have a reduced size of 512 instead of 2048, to reduce the size of the dataset on disk. Tiles from the WSI files were extracted and saved to disk as .png files, for pre-training the Resnet18 backbone directly on these tiles. ˜4 million random tiles were extracted to reduce the dataset size on disk, instead of extracting all of the tiles.

Random dataset extraction pointer:

Branch: feature/sample_tiles_per_slide_extraction Example:

sbatch -p M-48Cpu-371GB --array 1-8 scripts/dataset_extraction/extract_iris_dataset_mil.py --
iris_ --image_magnification <mag> --tile_dim 256 --step 256
--wsi_tile_filter_type atd --filter_ratio_threshold=0.99 --cont --iris_id=5239 --output_path
<output_path>
--tiles_per_slide 100

Creating Embeddings in 10× magnification:

sbatch --mem 70gb --array 0-7 --partition gpu --gres=gpu:1
scripts/embeddings/create_embeddings_wsi.py
--slides_dir /pstore/data/dspta/data/aimm/4934/slides/ --backbone_name resnet18 --
backbone_checkpoint_path
/pstore/data/dspta/data/aimm/checkpoints/3102980_Experiment/weights.best.h5 --output_dir
/pstore/data/dspta/data/aimm/4934/resnet18_tile_predictins_embeddings_10x --batch_size 128 -
-filter_type atd
--study_id 4934 --magnification 10 --extrapolation_tolerance 999 --iris_{—}
--analysis_masks_dir /pstore/data/dspta/data/aimm/4934/analysis_masks -cont

### Exploratory Data Analysis

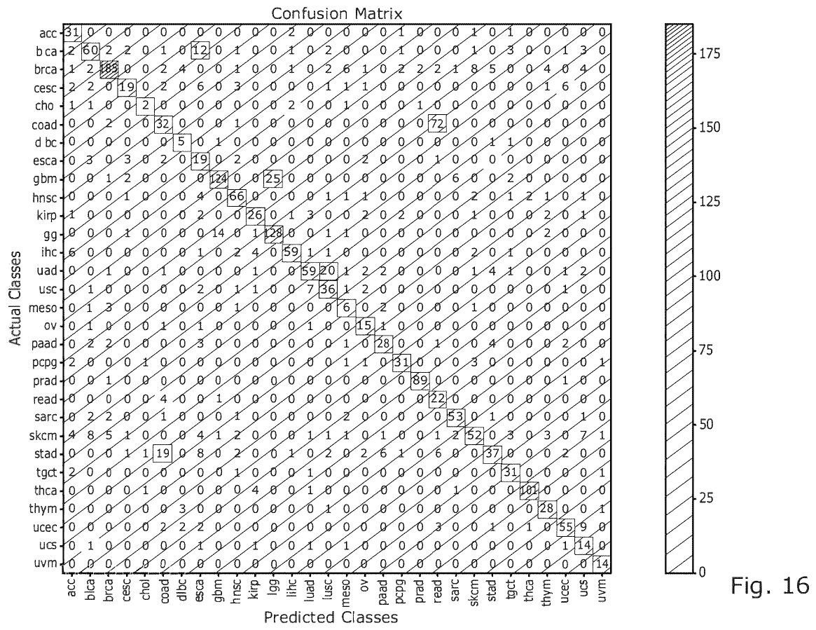

The UMAP visualization for the imagenet pretrained network embeddings (every point is the average tile embedding per slide) is shown in FIG. 16.

Note: the visualization was based on modifying this script:

scripts/general/umap_per_study.py
Branch: release/phase1b

### (And Modifying the Plot Title There)

Run command: python scripts/general/umap_per_study.py <json file with list of embedding files
per slide>

### Endpoint: Predicting 30 Cancer Types

Several methods were tested, namely Attention MIL on fixed ImageNet pretrained Resnet18 embeddings; Pretraining a Resnet18 classifier using noisy labels, on the tiles belonging to the dataset, and then aggregating the tile scores with mean pooling and Creating embeddings with the pretrained Resnet18 classifier, and then learning an Attention MIL model on the embeddings.

### Attention MIL on Fixed ImageNet Pretrained Resnet18 Embeddings

Results: 68% F1 score.

Confusion between LUAD/LUSC, READ/COAD etc. was expected: The per-category F1 scores are shown in Table 10.

TABLE 10

| Cancer Type | F1 |
|---|---|
| Chol | 0.3076923077 |
| Meso | 0.3157894737 |
| Read | 0.3259259259 |
| Coad | 0.367816092 |
| Esca | 0.3917525773 |
| Ucs | 0.4516129032 |
| Dlbc | 0.4545454545 |
| Cesc | 0.5135135135 |
| Stad | 0.524822695 |
| Lusc | 0.6 |
| Skcm | 0.6117647059 |
| Ov | 0.612244898 |
| Kirp | 0.6582278481 |
| Paad | 0.6746987952 |
| Blca | 0.6818181818 |
| Luad | 0.6900584795 |
| Acc | 0.7045454545 |
| Thym | 0.7567567568 |
| Ucec | 0.7586206897 |
| Tgct | 0.775 |
| Pcpg | 0.7948717949 |
| Hnsc | 0.8 |
| Lihc | 0.8194444444 |
| Gbm | 0.8239202658 |
| Sarc | 0.8412698413 |
| Brca | 0.8466819222 |
| Igg | 0.8504983389 |
| Uvm | 0.875 |
| Thca | 0.9528301887 |
| Prad | 0.9726775956 |

Training command:

sbatch --mem 200gb --partition gpu --gres gpu:4 --error
/pstore/data/dspta/data/aimm/logs/slurm.%j.err --output
/pstore/data/dspta/data/aimm/logs/slurm.% j.out scripts/weakly_supervised/trainers/train_mil.py
--epochs <epochs> --testing_frequency 1 --labels_cols_list “Cancer Type” --labels_file_path
/pstore/data/dspta/data/aimm/metadata/4934_all.csv --output_path
/pstore/data/dspta/data/aimm/checkpoints --batch_size <batch_size> --algorithm attention_mil
--bag_sample <bag_sample> --dropout <dropout> --kl_loss_weight <KL_weight> --
classes_names
‘coad:0,ov:1,thym:2,pcpg:3,blca:4,cesc:5,thca:6,luad:7,hnsc:8,lgg:9,ucec:10,stad:11,acc:12,tgc
t:13,kir
p:14,ucs:15,brca:16,lusc:17,meso:18,paad:19,sarc:20,skcm:21,prad:22,uvm:23,chol:24,lihc:25,
gbm:2
6,dlbc:27,esca:28,read:29’ --metric F1 --weight_decay 0.000 --lr 0.001 --embeddings
/pstore/data/dspta/data/aimm/jsons/4934_resnet18_10x_cancer_type.json --seed 0 --
test_slides_path
/pstore/data/dspta/data/aimm/splits/4934_resnet18_10x_cancer_type_test.txt --
percenttrain_data 0.8

Pretraining a Resnet18 classifier: using noisy labels on TCGA data, and then aggregating the tile scores with mean pooling Result: 80% F1 score. The label of every tile is the cancer type of the slide it belongs to. This is considered a “noisy” label, since some regions of the slides, e.g. fat regions, might not be informative of the cancer type and might be common to several cancer types. The model was trained for 30 epochs, sampling 128 random tiles per slide every epoch. The slide level prediction is then the average of all the predictions per category (after a softmax), and then taking the highest scoring category.

Creating embeddings with the TCGA-pretrained Resnet18 classifier, and then learning an Attention MIL model on the embeddings. 512 embeddings were extracted after the last convolutional layer from the resnet18 classifier, and then learn an Attention MIL network to aggregate the embeddings.

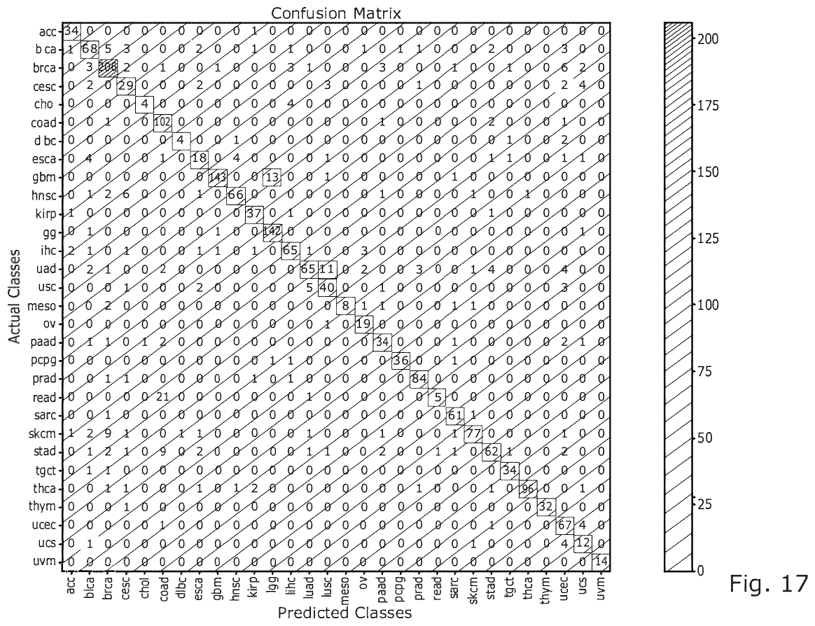

Result: 84% F1 score with the confusion matrix being shown in FIG. 17.

Training command:

sbatch --mem 200gb --partition gpu --gres gpu:4 --error
/pstore/data/dspta/data/aimm/logs/slurm.%j.err --output
/pstore/data/dspta/data/aimm/logs/slurm.%j.out scripts/weakly_supervised/trainers/train_mil.py
--epochs 100 --testing_frequency 1 --labels_cols_list “Cancer Type” --labels_file_path
/pstore/data/dspta/data/aimm/metadata/4934_all.csv --output_path
/pstore/data/dspta/data/aimm/checkpoints --batch_size 256 --algorithm attention_mil --
bag_sample
128 --dropout 0.65 --kl_loss_weight 1.2 --classes_names
‘coad:0,ov:1,thym:2,pcpg:3,blca:4,cesc:5,thca:6,luad:7,hnsc:8,lgg:9,ucec:10,stad:11,acc:12,tgc
t:13,kir
p:14,ucs:15,brca:16,lusc:17,meso:18,paad:19,sarc:20,skcm:21,prad:22,uvm:23,chol:24,lihc:25,
gbm:2
6,dlbc:27,esca:28,read:29’ --metric F1 --weight_decay 0.000 --lr0.001 --embeddings
/pstore/data/dspta/data/aimm/jsons/4934_resnet18_tile_prediction_10x_cancer_type.json --seed
0
--test_slides_path/pstore/data/dspta/data/aimm/splits/4934_resnet18_10x_cancer_type_test.txt
--percenttrain_data 0.8

Best epoch: 71

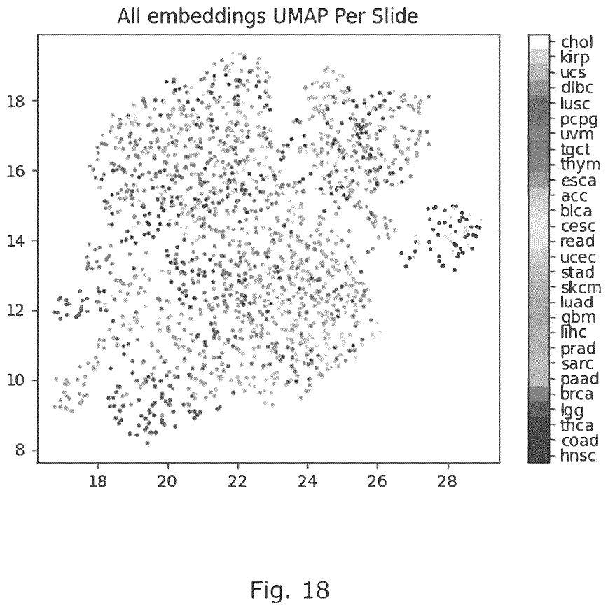

The UMAP visualization for the TCGA-pertrained network embeddings (every point is the average tile embedding per slide) being shown in FIG. 18.

Note: the visualization was based on modifying this script:

scripts/general/umap_per_study.py
Branch: release/phase1b

### (And Modifying the Plot Title Inside the Code)

Run command: python scripts/general/umap_per_study.py <json file with list of embedding files
per slide>

### Endpoint: Predicting the Primary Gleason Score

Attention-MIL was used on Resnet18 pre-trained Imagenet embeddings to predict the Gleason pattern for two scenarios i.e. Predicting Pattern 3 vs Pattern 4, and ignoring cases of Pattern 5 and Predicting Pattern 3 vs 4 vs 5. Gleason pattern 2 was omitted since it had only 1 case among the slides.

TABLE 11
is a breakdown of the Gleason Patterns in the dataset

| Pattern 3 | Pattern 4 | Pattern 5 | Pattern 2 |
|---|---|---|---|
| 174 | 243 | 31 | 1 |

### Results for Predicting Patterns 3/4/5

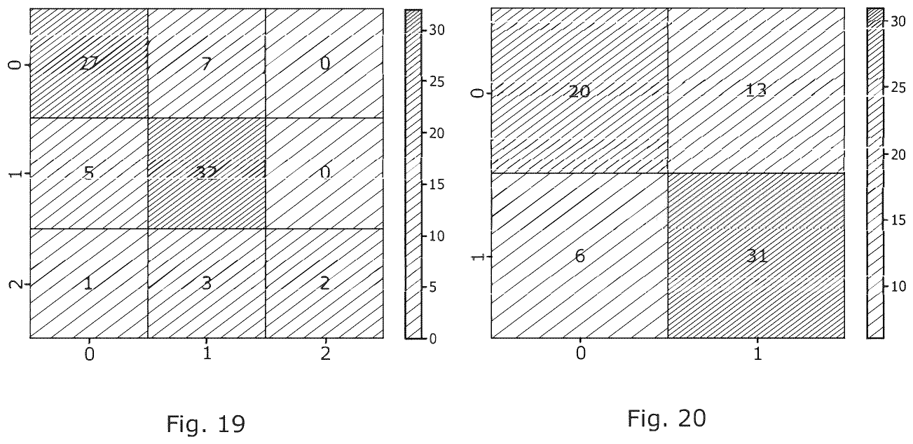

F1 score: 70.53% with the confusion matrix being shown in FIG. 19.

Training command:

sbatch --mem 200gb --partition gpu --gres gpu:4 --error
/pstore/data/dspta/data/aimm/logs/slurm.%j.err --output
/pstore/data/dspta/data/aimm/logs/slurm.%j.out scripts/weakly_supervised/trainers/train_mil.py
--epochs 100 --testing_frequency 1 --labels_cols_list “Primary Gleason Grade” --
labels_file_path
/pstore/data/dspta/data/aimm/metadata/4934_all.csv --output_path
/pstore/data/dspta/data/aimm/checkpoints --batch_size 256 --algorithm attention_mil --
bag_sample
256 --dropout 0.65 --kl_loss_weight 0.0 --classes_names ‘pattern 3:0,pattern 4:1,pattern 5:2’ --
metric
F1 --weight_decay 0.000 --lr 0.001 --embeddings
/pstore/data/dspta/data/aimm/jsons/4934_resnet18_tile_prediction_10x_primary_gleason_grade
.json --seed 0 --test_slides_path
/pstore/data/dspta/data/aimm/splits/primary_gleason_grade_pattern345_test.txt --
percenttrain_data
0.8

### Best Epoch: 92

Results for predicting Patterns 3 vs 4:

F1 score: 88% with the confusion matrix being shown in FIG. 20.

Training command:

sbatch --mem 200gb --partition gpu --gres gpu:4 --error
/pstore/data/dspta/data/aimm/logs/slurm.%j.err --output
/pstore/data/dspta/data/aimm/logs/slurm.%j.out scripts/weakly_supervised/trainers/train_mil.py
--epochs 100
--testing_frequency 1 --labels_cols_list “Primary Gleason Grade” --labels_file_path
/pstore/data/dspta/data/aimm/metadata/4934_all.csv --output_path
/pstore/data/dspta/data/aimm/checkpoints --batch_size 256 --algorithm
attention_mil_post_classifier --bag_sample 512 --dropout 0.65 --kl_loss_weight 0.0
--classes_names ‘pattern 3:0,pattern 4:1’ --metric F1 --weight_decay 0.000 --lr 0.001 --
embeddings
/pstore/data/dspta/data/aimm/jsons/4934_resnet18_tile_prediction_10x_primary_gleason_grade
.json --seed 0 --test_slides_path
/pstore/data/dspta/data/aimm/splits/primary_gleason_grade_pattern345_test.txt
--percenttrain_data 0.75

### Epoch: 69

### Multi Modal Prediction: Combining the Imaging and RNA Modalities

A combined prediction using both modalities was created. Some TCGA cases have only RNA data and not WSI data, or the other way around. The combined dataset is the subset of cases that have data from both modalities, and therefore it is reduced compared to using only the imaging. This explains the slight difference in the baseline Imaging model performance compared to the previous sections.

### Multi Modal Cancer Type Prediction

Late fusion: Multiplying the Probability scores of the models. In this method we multiply the category scores of the models. The RNA model here is from the RNA team, and is based on an sk-learn SVM with the next parameters: LinearSVC (C=10, penalty=‘11’,loss=‘squared_hinge’, class_weight=‘balanced’, dual=False,)

The RNA model performance is higher than the imaging model-0.952 vs 0.83 Macro F1. Combining the models by multiplying improves it to 95.8% F1. However, the improvement is very high for some of the categories. Table 12 is a breakdown of the per category performance. The highest improvements are in the BLCA, HNSC, LUSC, CESC and SARC categories.

TABLE 12

| | Genomic | Imaging | Multiplication |
|---|---|---|---|
| Avg. | 0.9516 | 0.8302 | 0.9579 |
| ACC | 1 | 0.8695 | 1 |
| BICA | 0.9452 | 0.7837 | 0.9733 |
| BRCA | 0.9931 | 0.9295 | 0.9953 |
| CESC | 0.8918 | 0.7228 | 0.9189 |
| CHOL | 0.8571 | 0.5714 | 0.8571 |
| COAD | 0.8942 | 0.6881 | 0.8942 |
| DLBC | 1 | 0.6153 | 1 |
| ESCA | 0.88 | 0.6538 | 0.88 |
| GBM | 1 | 0.878 | 1 |
| HNSC | 0.9571 | 0.9064 | 0.9787 |
| KIRP | 1 | 0.9189 | 1 |
| LGG | 1 | 0.9704 | 1 |
| LIHC | 0.9787 | 0.9064 | 0.9787 |
| LUAD | 0.9615 | 0.8322 | 0.9681 |
| LUSC | 0.9108 | 0.7663 | 0.9387 |
| MESO | 0.9565 | 0.7826 | 0.9565 |
| OV | 1 | 0.8387 | 1 |
| PAAD | 1 | 0.8354 | 1 |
| PCPG | 0.9841 | 0.9354 | 1 |
| PRAD | 1 | 0.9714 | 1 |
| READ | 0.5818 | 0.4935 | 0.5818 |
| SARC | 0.8529 | 0.9062 | 0.909 |
| SKCM | 0.9828 | 0.87 | 0.9885 |
| STAD | 0.9577 | 0.8549 | 0.9577 |
| TGCT | 1 | 0.9565 | 1 |
| THCA | 1 | 0.995 | 1 |
| THYM | 1 | 0.9387 | 1 |
| UCEC | 0.9624 | 0.8196 | 0.9624 |
| UCS | 1 | 0.6956 | 1 |
| UVM | 1 | 1 | 1 |

Code (repo: IRISAI Branch: mm):

python scripts/metrics/naive_multimodal.py --classes_names
“acc:0,blca:1,brca:2,cesc:3,chol:4,coad:5,dlbc:6,esca:7,gbm:8,hnsc:9,kirp:10,lgg:11
,lihc:12,luad:13,lusc:14,meso:15,ov:16,paad:17,pcpg:18,prad:19,read:20,sarc:21,skcm
:22,stad:23,tgct:24,thca:25,thym:26,ucec:27,ucs:28,uvm:29” --image_input_csv
/pstore/data/dspta/data/aimm/metadata/cancer_type_imaging_class_predictions.csv
--genomic_input_csv
/pstore/data/dspta/data/aimm/metadata/cancer_type_svm_class_prob_updated.csv
--testset_path
/pstore/data/dspta/data/aimm/splits/cancer_type_imaging_genomic_mm_testset.json
--num_of_categories 30

The Subset of Cases where Both Modalities Agree

If we look only at the subset of patients where both modalities agree with each other, The F1% is 98%, with the price of discarding 16% of the slides.

### Divergence In Correct Predictions (DCP):

To get a better understanding of the potential of the combination of the models the percentage of samples that the imaging model predicted correctly while the genomic model predicted incorrectly and vice versa (the genomic model predicted correctly while the imaging model predicted incorrectly) was calculated. The results implies that there is a potential:

$$DCP ( Genomic , Imaging ) = 12.1 \%$$

$$DCP ( Image , genomic ) = 2.5 \%$$

### Early Fusion: Concatenating the Raw Features of Both Modalities

A Neural Network was trained that combines the raw features. The first branch of the network processes the RNA features and reduces it to a 256 length vector. The second branch (that gets as an input the features from the resnet backbone, has a fully connected layer+ReLU non linearity), processes the imaging features and also reduces it to a 256 length vector. Then both vectors are concatenated, and are processed by several more fully connected layers to then predict 30 category types.

The Adam optimizer was used, and trained for 500 epochs, and the final epoch chosen based on the performance on the validation set (composed of 20% of the training set) as shown in FIG. 21.

Results:

Using this model only the RNA modality (keeping only the upper branch, and discarding the lower branch), we get 92% F1. Adding the lower branch, improves to 94% F1. It was noted that the training also converges much faster, in several tens of epochs instead of several hundreds. For the fixed model, adding both modalities improves over the RNA model. However it is still worse than the baseline SVM model that used a different dataset (only one data point per patient, vs several possible Whole Slide Images per patient in the imaging modality), and a different model. To further explore the benefits of Early Fusion, the model may need improvement, or modification to get the 95% F1 result using only the RNA data.

### Multi Modal Primary Gleason Score Prediction Late Fusion: Multiplying the Probability Scores of the Models

In this method the category scores of the models were multiplied.

Result for pattern 3/pattern 4 classification:

As shown in the table below, the imaging model performance is higher than the genomic model −0.72 vs 0.88 Macro F1.

Combining the models doesn't improve the results as shown in Table 13.

TABLE 13

| | Genomic | Imaging | Multiplication |
|---|---|---|---|
| Avg. | 0.724 | 0.8856 | 0.7821 |
| pattern 3 | 0.6885 | 0.8823 | 0.754 |
| pattern 4 | 0.7594 | 0.8888 | 0.8101 |

Code:

Model training command with IRISAI:

sbatch --mem 200gb --partition gpu --gres gpu:4 --error
/pstore/data/dspta/data/aimm/logs/slurm.%j.err --output
/pstore/data/dspta/data/aimm/logs/slurm.%j.out scripts/weakly_supervised/trainers/train_mil.py
--epochs 100
--testing_frequency 1 --labels_cols_list “Primary Gleason Grade” --labels_file_path
/pstore/data/dspta/data/aimm/metadata/4934_all.csv --output_path
/pstore/data/dspta/data/aimm/checkpoints
--batch_size 256 --algorithm attention_mil_post_classifier --bag_sample 512 --dropout 0.65 --
kl_loss_weight 0.0
--classes_names ‘pattern 3:0,pattern 4:1’ --metric F1 --weight_decay 0.000 --lr 0.001 --
embeddings
/pstore/data/dspta/data/aimm/jsons/4934_resnet18_tile_prediction_10x_primary_gleason_grade
.json --seed 0 --test_slides_path
/pstore/data/dspta/data/aimm/splits/primary_gleason_grade_pattern345_test.txt --
percenttrain_data 0.8
Multi-modal multiplication metrics command:
python scripts/metrics/naive_multimodal.py --classes_names ‘pattern 3:0,pattern 4:1’ --
image_input_csv
/pstore/data/dspta/data/aimm/metadata/34_imaging_primary_gleason_test_predictions.csv --
genomic_input_csv
/pstore/data/dspta/data/aimm/metadata/primary_gleason_genomic_autoML_19rfe_svm_class_p
redictions.csv
--testset_path
/pstore/data/dspta/data/aimm/splits/genomic_primary_gleason_testset_tcga_ids.json --
num_of_categories 2

Divergence In Correct Predictions (DCP):

To get a better understanding of the potential of the combination of the models, we calculated the percentage of samples that the imaging model predicted correctly while the genomic model predicted incorrectly and vice versa the genomic model predicted correctly while the imaging model predicted incorrectly. The results implies high potential:

$$DCP ( Genomic , Imaging ) = 15.71 \%$$

$$DCP ( Imaging , Genomic ) = 25.71 \%$$

Result for pattern 3/pattern 4/pattern 5 classification:

As shown in the table below, the imaging model performance is higher than the genomic model-0.67 vs 0.62 Macro F1. Combining the models (with confidence factors on the probabilities) by multiplying improves it to 0.7 F1.

Code:

Model training command with IRISAI:

sbatch --mem 200gb --partition gpu --gres gpu:4 --error
/pstore/data/dspta/data/aimm/logs/slurm.%j.err --output
/pstore/data/dspta/data/aimm/logs/slurm.%j.out scripts/weakly_supervised/trainers/train_mil.py
--epochs 100
--testing_frequency 1 --labels_cols_list “Primary Gleason Grade” --labels_file_path
/pstore/data/dspta/data/aimm/metadata/4934_all.csv --output_path
/pstore/data/dspta/data/aimm/checkpoints
--batch_size 256 --algorithm attention_mil --bag_sample 256 --dropout 0.65 --kl_loss_weight
0.0
--classes_names ‘pattern 3:0,pattern 4:1,pattern 5:2’ --metric F1 --weight_decay 0.000 --lr 0.001
--embeddings
/pstore/data/dspta/data/aimm/jsons/4934_resnet18_tile_prediction_10x_primary_gleason_grade
.json --seed 0 --test_slides_path
/pstore/data/dspta/data/aimm/splits/primary_gleason_grade_pattern345_test.txt --
percenttrain_data 0.8

Tune confidence factors on validation set command:

python scripts/metrics/tune_confidence_factor.py --classes_names ‘pattern 3:0,pattern 4:1,pattern
5:2’ --image_input_csv /pstore/data/dspta/data/aimm/metadata/345_val_predictions.csv --
genomic_input_csv
/pstore/data/dspta/data/aimm/metadata/val_xgb_class_prob_gleason_pattern345.csv --
num_of_categories 3

Predict on test set with confidence factors command:

```
python scripts/metrics/naive_multimodal.py --classes_names ‘pattern 3:0,pattern 4:1,pattern 5:2’
--image_input_csv /pstore/data/dspta/data/aimm/metadata/345_test_predictions.csv --
genomic_input_csv
/pstore/data/dspta/data/aimm/metadata/xgb_class_prob_gleason_pattern345.csv --
num_of_categories 3
--image_confidence_factor 0.88 --genomic_confidence_factor 0.04
  Locations of models
  Primary Gleason pattern 3/4/5:
/pstore/data/dspta/data/aimm/models/gs345_mag10_imaging.h5
  Primary Gleason pattern 3/4:
/pstore/data/dspta/data/aimm/models/gs34_mag10_imaging.h5
  Primary Gleason pattern 3/5:
/pstore/data/dspta/data/aimm/models/gs35_mag10_imaging.h5
  Primary Gleason pattern 4/5:
/pstore/data/dspta/data/aimm/models/gs45_mag10_imaging.h5
  LUAD/LUSC Survival Analysis:
/pstore/data/dspta/data/aimm/models/luad_lusc_survival_analysis_mag10_imaging.h 5
```

Cancer Type classification:
/pstore/data/dspta/data/aimm/models/cancer_type_mag10_imaging.h5

### Deploying the Models

For deploying/training the models, the “develop” branch in the IRISAI repository was used: https://bitbucket.org/rochedis/iris-ai/src. For the other python utility scripts described herein the multi modal branch called “mm” in this respiratory is used.

The command to deploy the model is:

python predict_on_wsis_attention.py --slides_dir <location_to_wsi_files>
--checkpoint_path <the MIL model>
--output_path <output directory to store the scores>
--batch_size 128
--heatmaps_data_path <folder to score the tile scores as .pkl files>
--wsi_tile_filter atd
--study_id 4934
--image_magnification 10
--extrapolation_tolerance 999
--step 256
--iris_url
--analysis_masks_dir masks
--tile_dim 256
--embedding_size512
--backbone_model_name resnet18 --filter_overlap_threshold=0.9

It is important to note that in the case of cancer type prediction, the backbone model should be the pretrained network.

Add:
-backbone_checkpoint_path <the cancer type backbone model>

Overall, using a cancer type classification learning model there was an accuracy of 84% using imaging data alone and 95.2% using genomics data alone. By combining the two models in a multi-model approach, the accuracy was increased to 98% but with 16% of the slides being rejected. For a Gleason score prediction model, imaging data alone gave a 70% F1 and a genomics data model alone gave a 64% accuracy. However, when the modalities are combined in a comparison of grade 3 vs 4 vs 5, and by rejecting slides where the two modalities disagree, the 70% accuracy rate increases to 90% F1. In a comparison of grades 3 vs 4 and again rejecting slides where the two modalities disagree (about 30% of the cases) the accuracy rate increases from 88% to 100%. This is shown in Table 14.

TABLE 14

| | | | | | | Gain of | |
|---|---|---|---|---|---|---|---|
| | | | | | Gain of | multi-modal | |
| | | Imaging | Genomics | | multi-modal | approach vs | Synergistic |
| | | (Histopathology) | (RNA-seq) | Imaging + Genomics | approach vs | RNA-seq | multi- |
| | | Best Specific | Best Specific | Best Specific | imaging | alone (per | modal |
| Modality | Class | F1 Score | Macro F1 score | Macro F1 score | (per class) | class) | effect |
| Cancer Type | | | | | | | |
| Adrenocortical | Acc | 0.87 | 0.98 | 1 | 13.05% | 2.00% | Yes |
| carcinoma | | | | | | | |
| Bladder | Blca | 0.78 | 0.95 | 0.97 | 19.48% | 2.39% | Yes |
| urothelial | | | | | | | |
| carcinoma | | | | | | | |
| breast invasive | Brca | 0.93 | 1 | 1 | 6.61% | −0.47% | |
| carcinoma | | | | | | | |
| cervical | Cesc | 0.72 | 0.88 | 0.92 | 21.34% | 4.34% | Yes |
| squamous cell | | | | | | | |
| carcinoma and | | | | | | | |
| endocervical | | | | | | | |
| adenocarcinoma | | | | | | | |
| Cholonglocarcinoma | Chol | 0.57 | 0.8 | 0.86 | 33.33% | 6.66% | Yes |
| Colon | Coad | 0.69 | 0.89 | 0.89 | 23.05% | 0.47% | |
| adenocarcinoma | | | | | | | |
| lymphoid | Dibc | 0.62 | 1 | 1 | 38.47% | 0.00% | |
| neoplasm | | | | | | | |
| diffuse large B | | | | | | | |
| cell lymphoma | | | | | | | |
| Esophageal | Esca | 0.65 | 0.86 | 0.88 | 25.70% | 2.27% | Yes |
| carcinoma | | | | | | | |
| Glioblastoma | Gbm | 0.88 | 1 | 1 | 12.20% | 0.00% | |
| multiforme | | | | | | | |
| head and neck | hnsc | 0.91 | 0.95 | 0.98 | 7.39% | 2.93% | Yes |
| squamous cell | | | | | | | |
| carcinoma | | | | | | | |
| kidney renal | Kirp | 0.92 | 1 | 1 | 8.11% | 0.00% | |
| papillary cell | | | | | | | |
| carcinoma | | | | | | | |
| brain lower | Lgg | 0.97 | 1 | 1 | 2.96% | 0.00% | |
| grade glioma | | | | | | | |
| liver | Lihc | 0.91 | 0.97 | 0.98 | 7.39% | 0.89% | Yes |
| hepatocellular | | | | | | | |
| carcinoma | | | | | | | |
| lung | Luad | 0.83 | 0.97 | 0.97 | 14.04% | −0.20% | |
| adenocarcinoma | | | | | | | |
| lung squamous | Lusc | 0.77 | 0.92 | 0.94 | 18.37% | 1.99% | Yes |
| cell carcinoma | | | | | | | |
| Mesothelioma | Meso | 0.78 | 0.96 | 0.96 | 18.18% | −0.37% | |
| Ovarian serous | Ov | 0.84 | 1 | 1 | 16.13% | 0.00% | |
| cystadenocarcinoma | | | | | | | |
| pancreatic | Paad | 0.84 | 1 | 1 | 16.46% | 0.00% | |
| adenocarcinoma | | | | | | | |
| Pheochromocytoma | Pcpg | 0.94 | 0.99 | 1 | 6.46% | 1.00% | Yes |
| and | | | | | | | |
| paraganglioma | | | | | | | |
| prostate | Prad | 0.97 | 1 | 1 | 2.86% | 0.00% | |
| adenocarcinoma | | | | | | | |
| rectum | Read | 0.49 | 0.6 | 0.58 | 15.18% | −3.13% | |
| adenocarcinoma | | | | | | | |
| Sarcoma | Sarc | 0.91 | 0.93 | 0.91 | 0.31% | −2.31% | |
| skin cutaneous | Skom | 0.87 | 0.97 | 0.99 | 11.99% | 1.87% | Yes |
| Melanoma | | | | | | | |
| stomach | Stad | 0.85 | 0.97 | 0.96 | 10.73% | −1.28% | |
| adenocarcinoma | | | | | | | |
| testicular germ | Tgct | 0.96 | 1 | 1 | 4.35% | 0.00% | |
| cell tumors | | | | | | | |
| thyroid | Thca | 1 | 1 | 1 | 0.50% | 0.00% | |
| carcinoma | | | | | | | |
| Thymoma | Thym | 0.94 | 1 | 1 | 6.13% | 0.00% | |
| uterine corpus | Ucec | 0.82 | 0.95 | 0.96 | 14.84% | 1.29% | Yes |
| endometrial | | | | | | | |
| carcinoma | | | | | | | |
| uterine | Ucs | 0.7 | 0.96 | 1 | 30.44% | 4.00% | Yes |
| carcinoma | | | | | | | |
| uterine | Uvm | 1 | 1 | 1 | 0.00% | 0.00% | |
| Melanoma | | | | | | | |
| Gleason score | | | | | | | |
| (Prostate | | | | | | | |
| Cancer) | | | | | | | |
| Gleason score | 3 | 0.88 | 0.67 | 0.75 | −17.02% | 11.14% | |
| 3 vs 4 | | | | | | | |
| Gleason score | 4 | 0.89 | 0.73 | 0.81 | −9.71% | 9.89% | |
| 3 vs 4 | | | | | | | |
| Gleason score | 3 | 0.79 | 0.67 | 0.81 | 1.46% | 16.86% | Yes |
| 3 vs 4 vs 5 | | | | | | | |
| Gleason score | 4 | 0.78 | 0.7 | 0.81 | 3.81% | 13.59% | Yes |
| 3 vs 4 vs 5 | | | | | | | |
| Gleason score | 5 | 0.44 | 0.55 | 0.5 | 11.12% | −10.00% | |
| 3 vs 4 vs 5 | | | | | | | |

The invention enables very powerful predictions and can be useful for a broad range of applications: from survival prediction, to predicting the cancer type and GleasonScore prediction.

The words “comprises/comprising” and the words “having/including” when used herein with reference to the present invention are used to specify the presence of stated features, integers, steps or components but does not preclude the presence or addition of one or more other features, integers, steps, components or groups thereof.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable sub-combination.

1. A method of diagnosing or determining the prognosis of cancer in a patient, the method comprising:
receiving genomic data of a patient;
receiving biopsy image data of the patient;
processing, using RNA-sequencing and a first machine learning model, the genomic data of the patient to determine at least one of a first cancer type or degree of cancer;
processing, using histopathology and a second machine learning model, the biopsy image data of the patient to determine at least one of a second cancer type or degree of cancer;
comparing the determined first type or degree of cancer with the determined second type or degree of cancer;
in response to determining a level of correlation between the determined first cancer type or degree and the second determined cancer type or degree, generating an output diagnosing or determining the prognosis of cancer in the patient as the first cancer type or degree;
in response to determining that the determined first cancer type or degree and the determined second cancer type or degree do not have the level of correlation, generating an output indicating that the diagnosing or determining the prognosis is undetermined.

2. The method of claim 1 wherein the first machine learning model comprises at least one of a support vector machine (SVM) or gradient boosting decision tree (GBDT).

3. The method of claim 1 wherein the second machine learning model comprises attention-based multiple instance learning (Attention MIL) or Resnet 18.

4. The method of claim 1 wherein the first machine learning model comprises a linear SVM model and the second machine learning model comprises a Resnet 18 model, and wherein generating an output diagnosis or determining the prognosis of a cancer comprises multiplying the probability scores of each of the first and second machine learning models.

5. The method of claim 1 wherein the genomic data is RNA sequence data.

6. The method of claim 5 wherein the RNA sequence data is derived from protein-encoding genes.

7. The method of claim 1 wherein the first and second cancer type or degree comprises at least one of cervical squamous cell carcinoma and endocervical adenocarcinoma (CESC), cholangiocarcinoma (CHOL), uterine carcinosarcoma (UCS), or Gleason score.

8. The method of claims 1 to 6 wherein the method is used to predict Luad/Lusc overall survival rate.

9. The method of claim 1 wherein determining the level of correlation comprises determining that the first and second types or degrees of cancer are the same and that an F1 score for the first machine learning model with respect to the first type or degree of cancer exceeds a first predetermined threshold and that an F1 score for the second machine learning model with respect the second type or degree of cancer exceeds a second predetermined threshold.

10. The method of claim 9 wherein the F1 score threshold is at least 90%.

11. A non-transitory computer-readable storage medium storing one or more computer programs configured to be executed by one or more processing units at a computer comprising instructions for:
receiving genomic data of a patient;
receiving biopsy image data of the patient;
processing, using RNA-sequencing and a first machine learning model, the genomic data of the patient to determine at least one of a first cancer type or degree of cancer;
processing, using histopathology and a second machine learning model, the biopsy image data of the patient to determine at least one of a second cancer type or degree of cancer;
comparing the determined first type or degree of cancer with the determined second type or degree of cancer;
in response to determining a level of correlation between the determined first cancer type or degree and the second determined cancer type or degree, generating an output diagnosing or determining the prognosis of cancer in the patient as the first cancer type or degree; or
in response to determining that the determined first cancer type or degree and the determined second cancer type or degree do not have the level of correlation, generating an output indicating that the diagnosing or determining the prognosis of is undetermined.

12. A computer system for diagnosing or determining the prognosis of cancer in a patient, the computer system comprising one or more processors, memory to store one or more computer programs, the computer programs comprising instructions for
receiving genomic data of a patient;
receiving biopsy image data of the patient;
processing, using RNA-sequencing and a first machine learning model, the genomic data of the patient to determine at least one of a first cancer type or degree of cancer;
processing, using histopathology and a second machine learning model, the biopsy image data of the patient to determine at least one of a second cancer type or degree of cancer;
comparing the determined first type or degree of cancer with the determined second type or degree of cancer;
in response to determining a level of correlation between the determined first cancer type or degree and the second determined cancer type or degree, generating an output diagnosing or determining the prognosis of cancer in the patient as the first cancer type or degree; or
in response to determining that the determined first cancer type or degree and the determined second cancer type or degree do not have the level of correlation, generating an output indicating that the diagnosing or determining the prognosis of is undetermined.

13. The system of claim 12 wherein the first machine learning model comprises at least one of a support vector machine (SVM) or gradient boosting decision tree (GBDT).

14. The system of claim 12 wherein the second machine learning model comprises attention-based multiple instance learning (Attention MIL) or Resnet 18.

15. The system of claim 12 wherein the first machine learning model comprises a linear SVM model and the second machine learning model comprises a Resnet 18 model, and wherein generating an output diagnosis or determining the prognosis of a cancer comprises multiplying the probability scores of each of the first and second machine learning models.

16. The system of claim 12 wherein the genomic data comprises RNA sequence data.

17. The system of claim 16 wherein the RNA sequence data is derived from protein-encoding genes.

18. The system of claim 12 wherein the first and second cancer type or degree comprises at least one of cervical squamous cell carcinoma and endocervical adenocarcinoma (CESC), cholangiocarcinoma (CHOL), uterine carcinosarcoma (UCS), or Gleason score.

19. The system of claim 12 wherein determining the level of correlation comprises determining that the first and second types or degrees of cancer are the same and that an F1 score for the first machine learning model with respect to the first type or degree of cancer exceeds a first predetermined threshold and that an F1 score for the second machine learning model with respect the second type or degree of cancer exceeds a second predetermined threshold.

20. The system of claim 19 wherein the F1 score threshold is at least about 90%.
